(19) United States
(12) Patent Application Publication
(10) Pub. No.: US 2022/0273780 A1
(43) Pub. Date: Sep. 1, 2022

(54) SELF-ASSEMBLED VACCINES AND COMBINATION THERAPIES FOR TREATING CANCER

(71) Applicant: The General Hospital Corporation, Boston, MA (US)

(72) Inventors: Mark C. Poznansky, Newton, MA (US); Jeffrey A. Gelfand, Cambridge, MA (US); Pierre R. LeBlanc, Haverhill, MA (US); Svetlana E. Korochkina, Lexington, MA (US)

(21) Appl. No.: 17/627,975

(22) PCT Filed: Jul. 17, 2020
(86) PCT No.: PCT/US20/42450

§ 371 Date: Jan. 18, 2022

Related U.S. Application Data
(60) Provisional application No. 62/876,045, filed on Jul. 19, 2019.

(51) Int. Cl. A61K 39/00; A61P 35/00; C07K 16/28; A61K 45/06

(52) U.S. Cl. CPC A61K 39/0011; A61P 35/00; C07K 16/2818; A61K 45/06; A61K 2039/5156; A61K 2039/505

(57) ABSTRACT

Provided herein are self-assembling pharmaceutical compositions comprising a heat shock protein fused to a biotin-binding protein, wherein the biotin-binding protein is non-covalently bound to a biotinylated component (e.g., tumor cell, tumor antigen, virus or viral antigen). The self-assembling pharmaceutical compositions may further comprise an immunotherapy (e.g., anti-PD-1 antibody). In addition, methods of using these pharmaceutical compositions to prevent and/or treat cancer, or to induce an immune response are provided. Methods of using the self-assembling pharmaceutical compositions in combination with an immunotherapy (e.g., anti-PD-1 antibody) are also provided.

### CROSS-REFERENCE TO RELATED APPLICATIONS

This application claims the benefit of U.S. Provisional Application No. 62/876,045, filed Jul. 19, 2019, which is incorporated by reference herein in its entirety.

### BACKGROUND

Though typically associated with infectious diseases, vaccines have a long history in the treatment of cancers. Similar to immunotherapy, cancer vaccines have had limited success and have not found widespread utility. Previously, cancer vaccines were based on the entire tumor, which can include signals from both healthy and cancerous cells, leading to less specific and lower overall activity. However, as DNA sequencing has become more widespread, it is now possible to identify mutations that are specifically associated with tumor cells and not found in healthy cells. Some tumor-specific mutations can serve as tumor-specific antigens or “neo-antigens”. These are new immune targets that can be used to train a patient's immune system to specifically target cancerous cells. Numerous companies are working to develop pipelines and algorithms to collect tumors from patients and identify targetable mutations than can be incorporated into personalized vaccines. However, identification of the targets is only the first challenge. The method by which the targets are presented to the immune system is equally, if not more, important than the actual targets. Vaccines that deliver good targets without appropriate immune stimulation can undermine any potential benefit.

Therefore, there is a need for a vaccine platform that can accommodate targets for any tumor type and that appropriately stimulates the expansion of specific anti-tumor immune cells.

### BRIEF SUMMARY

Provided herein are compositions and methods for preventing and/or treating cancer. In some aspects, provided herein are pharmaceutical compositions comprising, consisting essentially of, or consisting of a heat shock protein fused to a biotin-binding protein, wherein the biotin-binding protein is non-covalently bound to a biotinylated peptide, and wherein the peptide (1) binds to a MHC class I molecule; and (2) has less than 100% homology to an autologous native sequence and/or a native microbiome sequence. In some embodiment, the peptide is one or more peptides selected from Table 1 (on page 33). In some embodiments, the pharmaceutical composition is a vaccine. In some embodiments, the peptide has a length of 5-50 amino acids (e.g., a length of 8-12 amino acids).

In some aspects, provided herein are methods of preventing and/or treating ovarian cancer in a subject, comprising administering to the subject an effective amount of a pharmaceutical composition comprising a heat shock protein fused to a biotin-binding protein, wherein the biotin-binding protein is non-covalently bound to a biotinylated peptide and wherein the peptide (1) binds to a MHC class I molecule; and (2) has less than 100% homology to an autologous native sequence and/or a native microbiome sequence. In some embodiment, the peptide is one or more peptides selected from Table 1 (on page 33). In some embodiments, the peptide has a length of 5-50 amino acids (e.g., a length of 8-12 amino acids). In some embodiments, the pharmaceutical composition used in the methods described herein is a vaccine. In some embodiments, the method is a method of treating ovarian cancer (e.g., serous or epithelial papillary ovarian cancer). In certain embodiments, the pharmaceutical composition is administered to the subject as a non-covalent complex.

In some aspects, provided herein are pharmaceutical compositions comprising, consisting essentially of, or consisting of (1) a heat shock protein fused to a biotin-binding protein, wherein the biotin-binding protein is non-covalently bound to a biotinylated tumor cell or a biotinylated tumor antigen; and (2) an immunotherapy. In some embodiments, the tumor antigen binds to a MHC class I molecule. In some embodiments, the tumor antigen has less than 100% homology to an autologous native sequence and/or a native microbiome sequence. In some embodiments, the tumor antigen has a length of 5-50 amino acids (e.g., a length of 8-12 amino acids).

In some aspects, provided herein are methods of preventing and/or treating cancer in a subject, comprising administering to the subject an effective amount of a pharmaceutical composition comprising (1) a heat shock protein fused to a biotin-binding protein, wherein the biotin-binding protein is non-covalently bound to a biotinylated tumor cell or a biotinylated tumor antigen; and (2) an immunotherapy. In some embodiments, the tumor antigen binds to a MHC class I molecule. In some embodiments, the tumor antigen has less than 100% homology to an autologous native sequence and/or a native microbiome sequence. In some embodiments, the tumor antigen has a length of 5-50 amino acids (e.g., a length of 8-12 amino acids). In certain embodiments, the method is a method of treating cancer. In some embodiments, the biotinylated tumor cell or the biotinylated tumor antigen in the pharmaceutical composition described herein is derived from the same type of cancer as the cancer to be prevented and/or treated. For example, the cancer may be ovarian cancer (e.g., serous or epithelial papillary ovarian cancer), or Human Papilloma Virus (HPV)-related cancer (e.g., HPV-induced cervical cancer, HPV-induced anal cancer, or HPV-induced head and neck cancer). In some embodiments, the cancer is induced by infection of a tumor-producing virus (e.g., a Human Papillomavirus (HPV), Hepatitis C Virus (HCV), Epstein-Barr Virus (EBV), Human Immunodeficiency Virus (HIV), or Herpes virus). In some embodiments, the method further comprises a cancer therapy selected from the group consisting of radiation, a radiosensitizer, a chemotherapy, and a second immunotherapy. The immunotherapy or the second immunotherapy independently may be an immune checkpoint inhibitor or an immune modulator. In some embodiments, the immune modulator is a CXCR4/CXCR7 antagonist (e.g., AMD3100), a Jak/stat inhibitor (e.g., Ruxolitinib), or a near-infrared laser immunomodulation of skin-associated immune cell.

In some aspects, provided herein are methods of preventing and/or treating cancer in a subject, comprising conjointly administering to the subject an immunotherapy and an effective amount of a pharmaceutical composition comprising a heat shock protein fused to a biotin-binding protein, wherein the biotin-binding protein is non-covalently bound to a biotinylated tumor cell or a biotinylated tumor antigen. In some embodiments, the tumor antigen binds to a MHC class I molecule. In some embodiments, the tumor antigen has less than 100% homology to an autologous native sequence and/or a native microbiome sequence. In some embodiments, the tumor antigen has a length of 5-50 amino acids (e.g., a length of 8-12 amino acids). In some embodiments, the method is a method of treating cancer. In some embodiments, the immunotherapy and the pharmaceutical composition are administered concurrently or sequentially. In some embodiments, the pharmaceutical composition is administered before the immunotherapy. In some embodiments, the biotinylated tumor cell or the biotinylated tumor antigen in the pharmaceutical composition is derived from the same type of cancer as the cancer to be prevented or treated. For example, the cancer may be ovarian cancer (e.g., serous or epithelial papillary ovarian cancer), Human Papilloma Virus (HPV)-related cancer (e.g., HPV-induced cervical cancer, HPV-induced anal cancer, HPV-induced oral cancer, HPV-induced vulvar cancer, HPV-induced vaginal cancer, HPV-induced penile cancer, or HPV-induced head and neck cancer). In some embodiments, the cancer is induced by infection of a tumor-producing virus (e.g., a HPV, HCV, EBV, HIV, or Herpes virus). In some embodiments, the method further comprises a cancer therapy selected from the group consisting of radiation, a radiosensitizer, and a chemotherapy.

In some embodiments, the biotin-binding protein is non-covalently bound to a biotinylated tumor cell; and the biotinylated tumor cell expresses an antigen on its surface. In some embodiments, the tumor cell is non-replicative, for example, due to irradiation. In some embodiments, the biotinylated tumor cell is a biotinylated sarcoma cell or a biotinylated carcinoma cell, for example, the biotinylated tumor cell is a biotinylated fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemia, polycythemia Vera, lymphoma, multiple myeloma, Waldenstrom's macroglobulinemia, head and neck cancer, anal cancer, or heavy chain disease cell. In certain embodiments, the biotinylated tumor cell is a biotinylated ovarian cancer cell (e.g., a biotinylated serous or epithelial papillary ovarian cancer cell). In certain embodiments, the biotinylated tumor cell is a biotinylated Human Papilloma Virus (HPV)-related cancer cell (e.g., a biotinylated HPV-induced cervical cancer, a biotinylated HPV-induced anal cancer, or a biotinylated HPV-induced head and neck cancer).

In some embodiments, the biotin-binding protein is non-covalently bound to a biotinylated tumor antigen. The tumor antigen may be a protein that is overexpressed by a tumor cell, or an immunogenic fragment thereof. The tumor antigen may be a protein that is specifically mutated in a tumor cell, or an immunogenic fragment thereof. In some embodiments, the tumor antigen comprises a whole or partial inactivated tumor-producing virus. In other embodiments, the tumor antigen comprises a protein or an immunogenic fragment thereof that is derived from a tumor-producing virus. The tumor-producing virus may be, for example, HPV, HCV, EBV, HIV, or Herpes virus. In some embodiments, the tumor antigen is tumor-derived phospho-peptides. In some embodiments, the tumor antigen is capable of eliciting an immune response. In some embodiments, the tumor antigen is derived from a sarcoma cell or a carcinoma cell, for example, a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemias, polycythemia Vera, lymphoma, multiple myeloma, Waldenstrom's macroglobulinemia, head and neck cancer, anal cancer, or heavy chain disease cell. In certain embodiment, the tumor antigen is derived from an ovarian cancer cell (e.g., a serous or epithelial papillary ovarian cancer cell). In preferred embodiments, the tumor antigen is one or more peptides selected from Table 1 (on page 33). In certain embodiment, the tumor antigen is derived from a Human Papilloma Virus (HPV)-related cancer cell (e.g., a HPV-induced cervical cancer, HPV-induced anal cancer, HPV-induced oral cancer, HPV-induced vulvar cancer, HPV-induced vaginal cancer, HPV-induced penile cancer, or HPV-induced head and neck cancer).

In some embodiments, the immunotherapy inhibits an immune checkpoint. In certain embodiments, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. For example, the immune checkpoint may be PD1 or PD-L1. In preferred embodiments, the immunotherapy is an anti-PD-1 antibody. In some embodiments, the immunotherapy is an immune modulatory agent selected from the group consisting of a CXCR4/CXCR7 antagonist (e.g., AMD3100), a Jak/stat inhibitor (e.g., Ruxolitinib), and a near infrared laser immunomodulation of skin associated immune cell.

In some aspects, provided herein are methods of preventing and/or treating HPV-related cancer in a subject, comprising administering to the subject an effective amount of a pharmaceutical composition comprising a heat shock protein fused to a biotin-binding protein, wherein the biotin-binding protein is non-covalently bound to a biotinylated HPV virus or a biotinylated HPV viral antigen. In some embodiments, the HPV viral antigen binds to a MHC class I molecule. In some embodiments, the HPV viral antigen has less than 100% homology to an autologous native sequence and/or a native microbiome sequence. In some embodiments, the HPV viral antigen has a length of 5-50 amino acids (e.g., a length of 8-12 amino acids). In some embodiments, the pharmaceutical composition used in the methods described herein is a vaccine. In certain embodiments, the method is a method of treating HPV-related cancer (e.g., head and neck cancer or anal cancer). In some embodiments, the pharmaceutical composition is administered to the subject as a non-covalent complex.

In some aspects, provided herein are pharmaceutical compositions comprising, consisting essentially of, or consisting of (1) a heat shock protein fused to a biotin-binding protein, wherein the biotin-binding protein is non-covalently bound to a biotinylated HPV virus or a biotinylated HPV viral antigen; and (2) an immunotherapy. In some embodiments, the HPV viral antigen binds to a MHC class I molecule. In some embodiments, the HPV viral antigen has less than 100% homology to an autologous native sequence and/or a native microbiome sequence. In some embodiments, the HPV viral antigen has a length of 5-50 amino acids (e.g., a length of 8-12 amino acids).

In some aspects, provided herein are methods of preventing and/or treating HPV-related cancer in a subject, comprising administering to the subject an effective amount of a pharmaceutical composition comprising (1) a heat shock protein fused to a biotin-binding protein, wherein the biotin-binding protein is non-covalently bound to a biotinylated HPV virus or a biotinylated HPV viral antigen; and (2) an immunotherapy. In some embodiments, the HPV viral antigen binds to a MHC class I molecule. In some embodiments, the HPV viral antigen has less than 100% homology to an autologous native sequence and/or a native microbiome sequence. In some embodiments, the HPV viral antigen has a length of 5-50 amino acids (e.g., a length of 8-12 amino acids). In certain embodiments, the method is a method of treating HPV-related cancer (e.g., head and neck cancer or anal cancer). In some embodiments, the method further comprises a cancer therapy selected from the group consisting of radiation, a radiosensitizer, a chemotherapy, and a second immunotherapy. The immunotherapy or the second immunotherapy independently may be an immune checkpoint inhibitor or an immune modulator. In some embodiments, the immune modulator is a CXCR4/CXCR7 antagonist (e.g., AMD3100), a Jak/stat inhibitor (e.g., Ruxolitinib), or a near-infrared laser immunomodulation of skin-associated immune cells.

In some aspects, provided herein are methods of preventing and/or treating HPV-related cancer in a subject, comprising conjointly administering to the subject an immunotherapy and an effective amount of a pharmaceutical composition comprising a heat shock protein fused to a biotin-binding protein, wherein the biotin-binding protein is non-covalently bound to a biotinylated HPV virus or a biotinylated HPV viral antigen. In some embodiments, the HPV viral antigen binds to a MHC class I molecule. In some embodiments, the HPV viral antigen has less than 100% homology to an autologous native sequence and/or a native microbiome sequence. In some embodiments, the HPV viral antigen has a length of 5-50 amino acids (e.g., a length of 8-12 amino acids). In some embodiments, the method is a method of treating HPV-related cancer (e.g., head and neck cancer or anal cancer). In some embodiments, the immunotherapy and the pharmaceutical composition are administered concurrently or sequentially. In some embodiments, the pharmaceutical composition is administered before the immunotherapy. In some embodiments, the method further comprises a cancer therapy selected from the group consisting of radiation, a radiosensitizer, and a chemotherapy.

In some embodiments, the biotin-binding protein is non-covalently bound to a biotinylated HPV virus; and the biotinylated HPV virus expresses an antigen. The HPV virus may be a whole or partial inactivated HPV virus. In other embodiments, the biotin-binding protein is non-covalently bound to a biotinylated HPV viral antigen. The biotinylated HPV viral antigen may be biotinylated E6 protein, biotinylated E7 protein, or a biotinylated immunogenic fragment thereof. In specific embodiments, the biotinylated HPV viral antigen is selected from Table 3. In some embodiments, the pharmaceutical composition increases survival rate of subjects afflicted with HPV-related cancer (e.g., head and neck cancer or anal cancer).

Numerous embodiments are further provided that may be applied to any aspect of the present disclosure and/or combined with any other embodiment described herein. For example, in some embodiments, the biotin-binding protein is selected from the group consisting of avidin, streptavidin, and neutravidin. In some embodiments, the biotin-binding protein has an amino acid sequence that is at least 80%, 85%, 90%, 95%, or 99% identical to avidin or streptavidin.

In some embodiments, the heat shock protein is a mammalian heat shock protein or a bacterial heat shock protein. In certain embodiments, the heat shock protein is a member of the hsp70 family. In specific embodiments, the heat shock protein is or is derived from MTB-HSP70. For example, the heat shock protein may have an amino acid sequence that is at least 80%, 85%, 90%, 95%, or 99% identical to SEQ ID NO: 1 or SEQ ID NO: 2. In some embodiments, the pharmaceutical composition is a vaccine. In some embodiments, the pharmaceutical composition further comprises a pharmaceutically acceptable carrier. In some embodiments, the pharmaceutical composition increases survival rate of subjects afflicted with cancer (e.g., ovarian cancer such as serous or epithelial papillary ovarian cancer, or HPV-related cancer). In some embodiments, the pharmaceutical composition increases an immune response. In some embodiments, the pharmaceutical composition increases proliferation of immune cells.

In some aspects, provided herein are methods for producing a pharmaceutical composition described herein, comprising contacting a heat shock protein fused to a biotin-binding protein with a biotinylated peptide, sufficient to form a non-covalent complex of the heat shock protein and the biotinylated peptide, wherein the peptide (1) binds to a MHC class I molecule; and (2) has less than 100% homology to an autologous native sequence and/or a native microbiome sequence. In some embodiment, the peptide is one or more peptides selected from Table 1 (on page 33).

In some aspects, provided herein are methods of inducing an immune response in a subject, comprising administering to the subject an effective amount of a pharmaceutical composition described herein.

### BRIEF DESCRIPTION OF THE DRAWINGS

FIG. 1 shows a schematic diagram of the Self-Assembling Vaccine (SAV). MTB-HSP70 is the immune stimulating base unit for every SAV, regardless of target. The MTB-HSP70 includes Avidin to attach the variable unit, which provides the specific targeting peptide sequences (labelled variable unit).

FIG. 2 shows the percent survival of mice with different treatments.

### DETAILED DESCRIPTION

### General

The present invention is based at least in part on the discovery that a heat shock protein fusion in non-covalent association with a biotinylated component (e.g., tumor cell or tumor antigen) results in a Self-Assembling Vaccine (SAV) that increases percent of survival of mice that have cancer (e.g., ovarian cancer). Importantly, combinatory treatment of a SAV and an immunotherapy (e.g., anti-PD-1 antibody) showed a synergistic effect on prompting survival of these mice. These effects are at least in part due to the increased proliferation of immune cells. Accordingly, compositions and methods for preventing and/or treating cancer using a SAV alone or in combination with an immunotherapy, are provided.

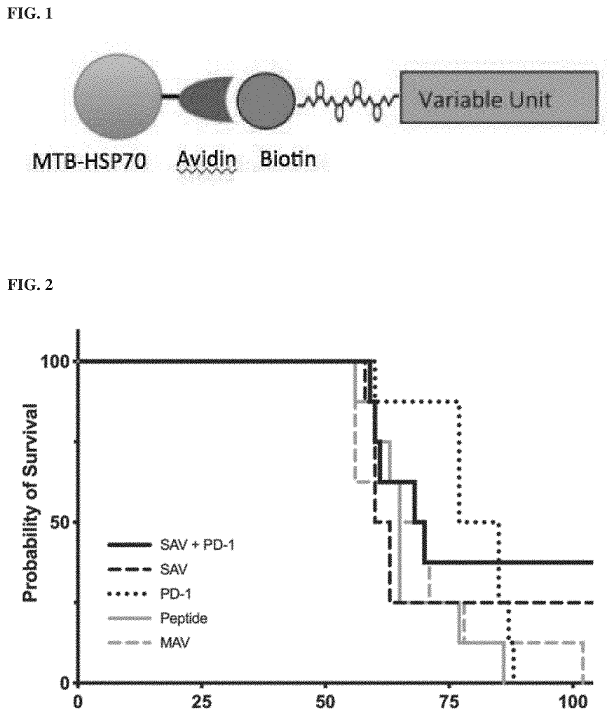

A major challenge for all vaccines is the cost and time required for development. These constraints are particularly acute in the context of emerging infectious diseases and cancer, where vaccines must be tailored to the target and then produced quickly and cost effectively. To address this gap, in certain embodiments, the invention relates to a modular platform with an immune activating base unit that can be coupled with targeting modules that elicit immune responses. Though originally developed to target infectious diseases, the platform was demonstrated herein to be equally capable of delivering targeting modules to different cancer types. In some embodiments, the base unit of the platform is MTB-HSP70, a bacterial protein with known immune stimulating properties, which is modified to include avidin. The incorporation of avidin allows the base to be linked to specific targeting modules, such as protein subunits called peptides (see FIG. 1). In certain embodiment, the peptides used to target the tumor can be selected from proteins that are abnormally abundant in the tumor, mutations that are unique to the tumor, and from modifications to proteins that are hallmarks of cancer cells. Computational tools can be used to identify target specific peptides that are predicted to provide good targets for the immune system, to determine the appropriate structure of the peptide chain, and to incorporate any changes needed to synthesize custom peptides.

In certain embodiments, these custom peptides include biotin, which binds to avidin, forming an incredibly stable linkage between the immune stimulating and cancer targeting components. This approach was termed a “self-assembling vaccine” or “SAY”, as it can be prepared without the need for any further specialized chemistry or purification. Previously, it has been shown that the SAV approach can promote specific immune responses against bacterial and viral targets (Leblanc et al. (2014) Human Vaccin. Immunother. 10:3022-3038). Other studies have also shown that MTB-HSP70 can improve the function of tumor targeting antibodies. Therefore, SAV technology may be used to create tumor-specific vaccines, which optionally further comprise an inbuilt broadly immune-activating adjuvant. In certain embodiments, the vaccines target tumors in a number of ways, resulting in a favorable anti-tumor immune response.

Ovarian cancer is a particularly urgent area of unmet need as the great majority of women diagnosed with the disease have late stage cancer. For most women, surgery and chemotherapy are initially effective. However, a large percentage of women ultimately relapse within five years. While immunotherapy has shown significant promise for other cancer types, the results in ovarian cancer have been underwhelming. The recent enthusiasm around immunotherapy has led to renewed interest in the development of cancer vaccines. Effective therapies for cancer will likely rely on a combination of the traditional approaches of surgery and chemotherapy together with regimens tailored to each patient that can include targeted drugs, immunotherapy, and personalized vaccines.

In certain embodiments, the SAV comprise a peptide from a protein known to be overexpressed by tumors or from a protein that is specifically mutated in the cancer cells. In certain embodiments, the peptide alone elicits an immune response.

In certain embodiments, the invention relates to a pharmaceutical composition co-administered with an immunotherapy agent, for example, an antibody that targets PD-1. Immune cells that have become exhausted in the fight against an infection or tumor often have high levels of PD-1 on their surface. The anti-PD-1 antibody can bind the surface of these low-functioning immune cells and re-invigorate them. However, this treatment does not increase the number of anti-tumor immune cells in the body. In certain embodiments, the use of a tumor-targeted vaccine (to expand the number of anti-cancer immune cells) together with anti-PD-1 (to restore and maintain their function) produces results superior to those seen to date with either approach alone.

### Definitions

For convenience, before further description of the present invention, certain terms employed in the specification, examples and appended claims are defined here.

The singular forms “a”, “an”, and “the” include plural references unless the context clearly dictates otherwise.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the present invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “administering” is intended to include routes of administration which allow an agent to perform its intended function. Examples of routes of administration for treatment of a body which can be used include injection (subcutaneous, intravenous, parenteral, intraperitoneal, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

“Parenteral” means modes of administration other than enteral and topical administration, usually by injection, and includes, without limitation, intravenous, intramuscular, intralesional, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection, oral, epidural, intranasal, and infusion.

As used herein, the term “conjoint administration” or “conjointly administered” or “co-administered” refers to any form of administration of two or more different agents such that the second agent is administered while the previously administered agent is still effective in the body (e.g., the two agents are simultaneously effective in the subject, which may include synergistic effects of the two agents). For example, the different agents can be administered either in the same formulation or in separate formulations, either concomitantly or sequentially. Thus, a subject who receives such treatment can benefit from a combined effect of different agents.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing. The names of the natural amino acids are abbreviated herein in accordance with the recommendations of IUPAC-IUB.

The term “antibody” refers to an immunoglobulin, or derivatives thereof, which maintain specific binding ability, and proteins having a binding domain which is homologous or largely homologous to an immunoglobulin binding domain. The term “antibody” is intended to encompass whole antibodies or antigen-binding fragments thereof. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. An antibody may be an engineered or naturally occurring antibody.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, Fc, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

An “antigen” refers to a target of an immune response induced by a composition described herein. An antigen may be a protein antigen and is understood to include an entire protein, fragment of the protein exhibited on the surface of a virus or an infected, foreign, or tumor cell of a subject as well as peptide displayed by an infected, foreign, or tumor cell as a result of processing and presentation of the protein, for example, through the typical MHC class I or II pathways. Examples of such foreign cells include bacteria, fungi, and protozoa.

In some embodiments, the “antigen” binds to a MHC class I molecule (e.g., a HLA molecule). Methods for testing the binding potential of antigens against HLA class II and class I alleles are well known in the art. For example, the HLA binding can be predicted using a publically known algorithm (e.g., EpiMatrix algorithm), or tested using standard in vitro HLA binding assays (e.g., a competition-based assay) as described in Scholzen et al. (2019) Frontiers in Immunology 10:1-22. The binding affinity may be measured by IC₅₀ using a competition-based assay. For example, proteins or peptides have IC₅₀ values of no more than 100 μM in an HLA class II binding assay may be considered as “binders”, while proteins or peptides have IC₅₀ values too high to accurately measure under binding conditions tested (>100 μM) or with no dose-dependent responses are considered non-binders. In an HLA class I binding assay, proteins or peptides have IC₅₀ values of no more than 1000 μM may be considered as “binders”, while proteins or peptides have IC₅₀ values too high to accurately measure under binding conditions tested (>1000 μM) or with no dose-dependent responses are considered non-binders.

In some embodiments, the “antigen” has no or minimal autoreactivity, and/or microbiome reactivity. “Autoreactivity” can be predicted based on the sequence homology shared between an antigen and an autologous native sequence. In some embodiments, the antigen has a sequence that is less than 100%, 99%, 95%, 90%, 85%, 80%, 75%, 70%, 65%, 60%, 55%, 50%, 45%, 40%, 35%, 30%, 25%, 20%, 15%, 10%, 5%, 1% homology to an autologous native sequence. “Microbiome reactivity” can be predicted based on the sequence homology shared between an antigen and a native microbiome sequence. In some embodiments, the antigen has a sequence that is less than 100%, 99%, 95%, 90%, 85%, 80%, 75%, 70%, 65%, 60%, 55%, 50%, 45%, 40%, 35%, 30%, 25%, 20%, 15%, 10%, 5%, 1% homology to a native microbiome sequence. In some embodiments, homology analysis can be done using publically known algorithm (e.g., JanusMatrix algorithm) as described in Scholzen et al. (2019) Frontiers in Immunology 10:1-22. In some embodiments, the “antigen” is a peptide that has a length of from about 5 to about 100 amino acids, for example, from about 5 to about 90, from about 5 to about 80, from about 5 to about 70, from about 5 to about 60, from about 5 to about 50, from about 5 to about 45, from about 5 to about 40, from about 5 to about 35, from about 5 to about 30, from about 5 to about 25, from about 5 to about 20, from about 5 to about 15, or from about 8 to about 12 amino acids. In specific embodiments, the “antigen” is a peptide that has a length of 5, 6, 7, 8, 9, 10, 11, 12, 13, 14, 15, 16, 17, 18, 19, 20, 21, 22, 23, 24, or 25 amino acids. Examples of bacterial antigens include Protein A (PrA), Protein G (PrG), and Protein L (PrL). Examples of tumor antigens include, but are not limited to, peptides listed in Table 1 (on page 33). Examples of viral antigens include, but are not limited to, peptides listed in Table 3 (on page 38).

The term “antigen binding site” refers to a region of an antibody that specifically binds an epitope on an antigen.

The term “biotin-binding protein” refers to a protein, which non-covalently binds to biotin. A biotin-binding protein may be a monomer, dimer, or tetramer, capable of forming monovalent, divalent, or tetravalent pharmaceutical compositions, respectively, as described herein. Non-limiting examples include anti-biotin antibodies, avidin, streptavidin, and neutravidin. The avidin may comprise mature avidin, or a sequence that is at least 80%, 85%, 90%, 95%, or 99% identical to the sequence identified by NCBI Accession No. NP_990651. The streptavidin may comprise, for example, a sequence that is at least 80%, 85%, 90%, 95%, or 99% identical to the sequence identified by of NCBI Accession No. AAU48617. The term “biotin-binding protein” is intended to encompass wild-type and derivatives of avidin, streptavidin, and neutravidin, which form monomers, dimers or tetramers. Examples of such derivatives are set forth below and also described in Laitinen, 0. H. (2007), “Brave New (Strept)avidins in Biotechnology,” Trends in Biotechnology 25 (6): 269-277 and Nordlund, H. R. (2003), “Introduction of histidine residues into avidin subunit interfaces allows pH-dependent regulation of quaternary structure and biotin binding,” FEBS Letters 555: 449-454, the contents of both of which are expressly incorporated herein by reference.

The term “tumor cell” when used in the context as an antigen-containing biotinylated component is intended to encompass whole tumor cells or portions thereof, provided that the portions contain the antigen of interest on a surface accessible for recognition by the immune system when a pharmaceutical composition comprising the biotinylated “tumor cell” is administered to a subject.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “HPV-related cancer” or “HPV-associated cancer” as used herein refers to any type of cancer that is associated with or is caused by Human Papillomavirus (HPV) infection. Persistent infection of certain HPV types (e.g., types 16, 18, 31 and 45) has been linked to cancers such as cancer of the oropharynx, larynx, vulva, vagina, cervix, penis, and anus. In some embodiments, HPV-related cancer may include, but is not limited to, cervical cancer, head and neck cancer, oral cancer, anal cancer, vulvar cancer, vaginal cancer, penile cancer, lung cancer, and oropharyngeal cancer. In specific embodiments, HPV-related cancer is cervical cancer, head and neck cancer, or anal cancer.

The terms “comprise” and “comprising” are used in the inclusive, open sense, meaning that additional elements may be included.

The term “costimulatory molecule” as used herein includes any molecule which is able to either enhance the stimulating effect of an antigen-specific primary T cell stimulant or to raise its activity beyond the threshold level required for cellular activation, resulting in activation of naive T cells. Such a costimulatory molecule can be a membrane-resident receptor protein.

The term “effective amount” refers to that amount of a pharmaceutical composition which is sufficient to effect a desired result. An effective amount of a pharmaceutical composition can be administered in one or more administrations.

The phrases “therapeutically-effective amount” and “effective amount” as used herein mean the amount of an agent which is effective for producing the desired therapeutic effect in at least a sub-population of cells in a subject at a reasonable benefit/risk ratio applicable to any medical treatment.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY). Examples of engineered antibodies include enhanced single chain monoclonal antibodies and enhanced monoclonal antibodies. Examples of engineered antibodies are further described in PCT/US2007/061554, the entire contents of which are incorporated herein by reference.

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

A “fusion protein” refers to a hybrid protein which comprises sequences from at least two different proteins. The sequences may be from proteins of the same or of different organisms. In various embodiments, the fusion protein may comprise one or more amino acid sequences linked to a first protein. In the case where more than one amino acid sequence is fused to a first protein, the fusion sequences may be multiple copies of the same sequence, or alternatively, may be different amino acid sequences. A first protein may be fused to the N-terminus, the C-terminus, or the N- and C-terminus of a second protein.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)₂ fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain. “Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc.), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc. “Host cell” refers to a cell that may be transduced with a specified transfer vector.

The cell is optionally selected from in vitro cells such as those derived from cell culture, ex vivo cells, such as those derived from an organism, and in vivo cells, such as those in an organism. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “immunogenic” refers to the ability of a substance to elicit an immune response. An “immunogenic composition,” or “immunogen” is a composition or substance which elicits an immune response. An “immune response” refers to the reaction of a subject to the presence of an antigen, which may include at least one of the following making antibodies, developing immunity, developing hypersensitivity to the antigen, and developing tolerance. In specific embodiments, the “immune response” refers to an anti-tumor immune response.

The term “including” is used herein to mean “including but not limited to”. “Including” and “including but not limited to” are used interchangeably.

A “linker” is art-recognized and refers to a molecule or group of molecules connecting two covalent moieties, such as a heat shock protein and biotin-binding protein. The linker may be comprised of a single linking molecule or may comprise a linking molecule and a spacer molecule, intended to separate the linking molecule and a moiety by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “multivalent” when in reference to a self-assembling pharmaceutical composition described herein refers to a heat shock fusion protein that is non-covalently bound to more than one biotinylated component. The term “divalent” when in reference to a self-assembling pharmaceutical composition described herein refers to a heat shock fusion protein that is non-covalently bound to two biotinylated components (e.g., tumor cells or tumor antigens). The term “tetravalent” when in reference to a self-assembling pharmaceutical composition described herein refers to a heat shock fusion protein that is non-covalently bound to four biotinylated components (e.g., tumor cells or tumor antigens). The biotinylated components (e.g., tumor cells or tumor antigens) of a multivalent pharmaceutical composition may have identical or different identities.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

A “patient” or “subject” or “host” are used interchangably, and each refers to either a human or non-human animal. This term includes mammals such as humans, primates, livestock animals (e.g., bovines, porcines), companion animals (e.g., canines, felines) and rodents (e.g., mice, rabbits and rats).

The phrase “pharmaceutically acceptable” is employed herein to refer to those pharmaceutical compositions which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” as used herein means a pharmaceutically-acceptable material, composition or vehicle, such as a liquid or solid filler, diluent, excipient, or solvent encapsulating material, involved in carrying or transporting the subject pharmaceutical composition from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Unless the context clearly indicates otherwise, “protein,” “polypeptide,” and “peptide” are used interchangeably herein when referring to a gene expression product, e.g., an amino acid sequence as encoded by a coding sequence. A “protein” may also refer to an association of one or more proteins, such as an antibody. A “protein” may also refer to a protein fragment. A protein may be a post-translationally modified protein such as a glycosylated protein. By “gene expression product” is meant a molecule that is produced as a result of transcription of an entire or part of a gene. Gene products include RNA molecules transcribed from a gene, as well as proteins translated from such transcripts. Proteins may be naturally occurring isolated proteins or may be the product of recombinant or chemical synthesis. The term “protein fragment” refers to a protein in which amino acid residues are deleted as compared to the reference protein itself, but where the remaining amino acid sequence is usually identical to that of the reference protein. Such deletions may occur at the amino-terminus or carboxy-terminus of the reference protein, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. Fragments of may be obtained using proteinases to fragment a larger protein, or by recombinant methods, such as the expression of only part of a protein-encoding nucleotide sequence (either alone or fused with another protein-encoding nucleic acid sequence). In various embodiments, a fragment may comprise an enzymatic activity and/or an interaction site of the reference protein to, e.g., a cell receptor. In another embodiment, a fragment may have immunogenic properties. The proteins may include mutations introduced at particular loci by a variety of known techniques, which do not adversely effect, but may enhance, their use in the methods provided herein. A fragment can retain one or more of the biological activities of the reference protein.

The term “self-assembling” as used herein refers to the ability of a heat shock protein fused to a biotin-binding protein to form a non-covalent complex with biotinylated component(s) as described herein. Such ability is conferred by the non-covalent association of biotin with a biotin-binding protein.

The term “single chain variable fragment” or “scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

“Treating” a disease in a subject or “treating” a subject having a disease refers to subjecting the subject to a pharmaceutical treatment, e.g., the administration of a drug, such that the extent of the disease is decreased or prevented. Treatment includes (but is not limited to) administration of a composition, such as a pharmaceutical composition, and may be performed subsequent to the initiation of a pathologic event.

As used herein, a therapeutic that “prevents” a condition (e.g., cancer) refers to a composition that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

The term “vaccine” refers to a pharmaceutical composition that elicits an immune response to an antigen of interest. The vaccine may also confer protective immunity upon a subject.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more cancer agents (e.g., a pharmaceutical composition described herein in combination with immunotherapy) can be greater than the sum of the separate effects of the cancer agents/therapies alone.

The term “phospho-peptide” refers to a phosphorylated peptide that can induce an immune response. The peptide may be phosphorylated at serine, threonine or tyrosine residues. In some embodiments, the phospho-peptide is derived from a cancer cell and can induce anti-tumor immune response.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

### Biotinylated Components

The term “biotinylated component” as used herein, refers to a biotinylated protein, cell, or virus. Non-limiting examples of biotinylated components include biotinylated tumor antigens, tumor cells, and costimulatory molecules. The biotinylated component (e.g., tumor cell, tumor antigen, virus, or viral antigen) is to be administered to a subject in conjunction with a heat shock protein fusion as described herein.

In one embodiment, the biotinylated tumor cell or tumor antigen is derived from a subject, which may be the same or a different person to whom the pharmaceutical compositions are to be administered. For example, a tumor cell or tumor antigen to which an immune response is desired can be isolated from a subject and optionally be amplified or cloned in vitro. The tumor cell or tumor antigen may then be biotinylated in vitro using methods known in the art. The biotinylated tumor cell or tumor antigen may then be administered in conjunction with a heat shock protein fusion described herein to the identical subject from which the tumor cell or tumor antigen was isolated, thus allowing for the development of personalized vaccines. Alternatively, the biotinylated tumor cell or tumor antigen may be administered in conjunction with a heat shock protein fusion described herein to a different subject from which the tumor cell or tumor antigen was isolated. The latter approach allows for the development of vaccines for the general population against cancer when administered to a general population.

Both approaches provide distinct advantages over the art, namely that, the tumor cell or tumor antigen need only be identified to the extent that allows for its correlation to a specific cancer and allows for its isolation from the subject. Such is a new approach for targeting antigens whose sequence may not be known or structure even identified. Thus, the present invention allows for the preparation of pharmaceutical compositions to induce an immune response to known or unidentified, uncharacterized antigen or antigens. Personalized vaccines provide an additional advantage over conventional vaccines in that HLA restriction is not problematic because the tumor cell or tumor antigen is derived from the identical host that the biotinylated tumor cell or tumor antigen is to be administered.

In some embodiments, the tumor cell or tumor antigen may be derived from a cancer cell line.

The tumor cell or tumor antigen may be derived from derived from the same type of cancer as the cancer that is prevented and/or treated with the pharmaceutical compositions described herein. The tumor cell or tumor antigen may be derived from a cancer that is a different type from the cancer that is prevented and/or treated with the pharmaceutical compositions described herein. The tumor cell or tumor antigen may be derived from a cancer that has the same genetic mutations as the cancer that is prevented and/or treated with the pharmaceutical compositions described herein. The tumor cell or tumor antigen may be derived from a cancer that has different genetic mutations from the that is prevented and/or treated with the pharmaceutical compositions described herein.

Any tumor cell or tumor antigen may be biotinylated and administered to a subject in conjunction with a heat shock protein fusion moiety described herein, such that the biotinylated tumor cell or tumor antigen when administered in conjunction with a heat shock fusion protein described herein elicits an anti-tumor immune response.

a. Biotinylated Tumor Cells

In some embodiments, a tumor cell is biotinylated and administered in conjunction with a heat shock protein fusion described herein. The tumor cell may be isolated from a subject. Isolation and purification of tumor cell from various tumor tissues such as surgical tumor tissues, ascites or carcinous hydrothorax is a common process to obtain the purified tumor cells. Cancer cells may be purified from fresh biopsy samples from cancer patients or animal tumor models. The biopsy samples often contain a heterogeneous population of cells that include normal tissue, blood, and cancer cells. Preferably, a purified cancer cell composition can have greater than 70%, 75%, 80%, 85%, 90%, 95%, 99%, or more, or any range in between or any value in between, total viable cancer cells. To purify cancer cells from the heterogeneous population, a number of methods can be used.

In one embodiment, laser microdissection is used to isolate cancer cells. Cancer cells of interest can be carefully dissected from thin tissue slices prepared for microscopy. In this method, the tissue section is coated with a thin plastic film and an area containing the selected cells is irradiated with a focused infrared laser beam pulse. This melts a small circle in the plastic film, causing cell binding underneath. Those captured cells are removed for additional analysis. This technique is good for separating and analyzing cells from different parts of a tumor, which allows for a comparison of their similar and distinct properties. It was used recently to analyze pituitary cells from dissociated tissues and from cultured populations of heterogeneous pituitary, thyroid, and carcinoid tumor cells, as well as analyzing single cells found in various sarcomas.

In another embodiment, fluorescence activated cell sorting (FACS), also referred to as flow cytometry, is used to sort and analyze the different cell populations. Cells having a cellular marker or other specific marker of interest are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well-known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations. One of the most common uses of this technology is for isolating circulating tumor cells (CTCs) from the blood of breast, NSC lung cancer, prostate and colon cancer patients using an antibody against EpCAM, a cell surface glycoprotein that has been found to be highly expressed in epithelial cancers.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

In still another embodiment, microfluidics, one of the newest technologies, is used to isolate cancer cells. This method used a microfluidic chip with a spiral channel that can isolate circulating tumor cells (CTCs) from blood based upon their size. A sample of blood is pumped into the device and as cells flow through the channel at high speeds, the inertial and centrifugal forces cause smaller cells to flow along the outer wall while larger cells, including CTCs, flow along the inner wall. Researchers have used this chip technology to isolate CTCs from the blood of patients with metastatic lung or breast cancer.

Fluorescent nanodiamonds (FNDs), according to a recently published article (Lin et al. Small (2015) 11:4394-4402), can be used to label and isolate slow-proliferating/quiescent cancer stem cells, which, according to study authors, have been difficult to isolate and track over extended time periods using traditional fluorescent markers. It was concluded that nanoparticles do not cause DNA damage or impair cell growth, and that they outperformed EdU and CFSE fluorescent labels in terms of long-term tracking capability.

It is to be understood that the purification or isolation of cells also includes combinations of the methods described above. A typical combination may comprise an initial procedure that is effective in removing the bulk of unwanted cells and cellular material. A second step may include isolation of cells expressing a marker common to one or more of the progenitor cell populations by immunoadsorption on antibodies bound to a substrate. An additional step providing higher resolution of different cell types, such as FACS sorting with antibodies to a set of specific cellular markers, may be used to obtain substantially pure populations of the desired cells.

In some other embodiments, the cancer cells are derived from a cancer cell line.

The tumor cell prior to introduction or reintroduction into a subject in the present invention is to be treated such that the cell no longer reproduces and causes harm to the subject to which it is administered. In some embodiments, the tumor cells are non-replicative. In certain embodiments, the tumor cells are non-replicative due to irradiation (e.g., γ and/or UV irradiation), and/or administration of an agent rendering cell replication incompetent (e.g., compounds that disrupt the cell membrane, inhibitors of DNA replication, inhibitors of spindle formation during cell division, etc.). In some embodiments, a sub-lethal dose of irradiation may be used. For example, the tumor cells may be sublethally irradiated before or after biotinylation to suppress cell proliferation prior to administration of the self-assembling vaccine to reduce the risk of giving rise to new neoplastic lesions. It is understood that irradiation is only one way to render the cells non-replicative, and that other methods which result in cancer cells incapable of cell division but that retain the ability to trigger the antitumor immunity are included in the present invention.

In some embodiments, the tumor cell expresses antigen on its surface, the identity of which may or may not be known or characterized. When administered to a subject in conjunction with the heat shock protein fusion, the non-covalent complex induces an immune response to the tumor antigen on the tumor cells. In some embodiments, the immune response is a “cytotoxic T cell” response against the tumor cell-expressing antigen, thereby targeting the tumor cells for destruction.

The tumor cell may be a cell of a type of cancer to be treated or prevented by the methods of the present invention. Such cells include, but are not limited to, for example, a human sarcoma cell or carcinoma cell, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, or heavy chain disease cell.

In some embodiments, the biotinylated tumor cell is a biotinylated ovarian cancer cell (e.g., serous or epithelial papillary ovarian cancer cell). In some embodiments, the biotinylated tumor cell is a biotinylated HPV-related cancer cell (e.g., a Human Papilloma Virus (HPV)-induced cervical cancer, HVP-induced head and neck cancer, or HVP-induced anal cancer).

b. Biotinylated Tumor Antigens

In some embodiments, a tumor antigen is biotinylated and administered in conjunction with a heat shock protein fusion described herein. An “antigen” refers to a target of an immune response induced by a composition described herein. An antigen may be a protein antigen and is understood to include an entire protein, fragment of the protein exhibited on the surface of a virus or an infected, foreign, or tumor cell of a subject as well as peptide displayed by an infected, foreign, or tumor cell as a result of processing and presentation of the protein, for example, through the typical MHC class I or II pathways. Examples of such foreign cells include bacteria, fungi, and protozoa.

In some embodiments, the “tumor antigen” of the present invention encompasses a tumor-associated protein and any portion or peptide of the tumor-associated protein capable of eliciting an anti-tumor response in a subject. The tumor antigen may be a protein that is overexpressed by a tumor cell, or an immunogenic fragment thereof. It may be a protein that is specifically mutated in a tumor cell, or an immunogenic fragment thereof. In certain embodiments, the tumor antigen is tumor-derived phospho-peptides. The tumor antigen can be any tumor-associated protein, fragment of the protein, modified form of the protein (e.g., phosphorylated protein or peptide), or functionally equivalent variant of the protein that is capable of eliciting an immune response. “Functionally equivalent variants” includes, but are not limited to, peptides with partial sequence homology, peptides having one or more specific conservative and/or non-conservative amino acid changes, peptide conjugates, chimeric proteins, fusion proteins and peptide nucleic acids.

The tumor antigen may be an antigen associated with a type of cancer to be treated or prevented by the methods of the present invention. In some embodiment, the tumor antigen is associated with sarcoma or carcinoma, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemias, polycythemia Vera, lymphoma, multiple myeloma, Waldenstrom's macroglobulinemia, head and neck cancer, anal cancer, or heavy chain disease.

In certain embodiments, the tumor antigen is associated with ovarian cancer (e.g., serous or epithelial papillary ovarian cancer). In some embodiments, the tumor antigen is associated with HPV-related cancer. In certain embodiments, the tumor antigen is associated with cervical cancer (e.g., a Human Papilloma Virus (HPV)-induced cervical cancer). In certain embodiments, the tumor antigen is associated with head and neck cancer (e.g., HPV induced head and neck cancer). In certain embodiments, the tumor antigen is associated with anal cancer (e.g., HPV induced anal cancer).

In some embodiments, the tumor antigen comprises a whole or partial inactivated tumor-producing virus. In some embodiments, the tumor antigen comprises a protein or an immunogenic fragment thereof that is derived from a tumor-producing virus. The tumor-producing virus may be, for example, HPV, HCV, EBV, HIV, or Herpes virus.

In certain embodiments, the tumor antigen is a peptide derived from a tumor-associated protein. As used herein the term “peptide” refers to native peptides (either degradation products or synthetically synthesized peptides) and further to peptidomimetics, such as peptoids and sernipeptoids which are peptide analogs, which may have, for example, modifications rendering the peptides more stable while in a body, or more immunogenic. Such modifications to include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified in Quantitative Drug Design, C. A Ramsden Gd., Chapter 17.2, F. Choplin Pergamon Press (1992), which is incorporated by reference in its entirety.

As used herein the term “derived from a protein” refers to peptides derived from the specified protein or proteins and further to homologous peptides derived from equivalent regions of proteins homologous to the specified proteins of the same or other species, provided that these peptides are effective as anti-tumor vaccines. The term further relates to permissible amino acid alterations and peptidomimetics designed based on the amino acid sequence of the specified proteins or their homologous proteins.

In certain embodiments, the peptides used to target the tumor may be selected from proteins that are abnormally abundant in the tumor, mutations that are unique to the tumor, and/or from modifications to proteins that are hallmarks of cancer cells. The peptides may be identified by DNA sequencing or by the literature. Computational tools may be used to identify target specific peptides that are predicted to provide good targets for the immune system, to determine the appropriate structure of the peptide chain, and/or to incorporate any changes needed to synthesize custom peptides. For example, neoantigens and tumor associated antigens identified by exomic DNA sequencing of the tumor cells may be selected by algorithmic analysis. The immunogenic peptides may be up-selected for computer predicted specific HLA binding and down-selected for autoreactivity, microbiome reactivity and/or immune suppressive activity using computer based algorithms (e.g., EpiMatrix algorithm or JanusMatrix algorithm). The HLA binding of predicted peptides may also be tested in standard peptide HLA binding in vitro assays as described in Scholzen et al. (2019) Frontiers in Immunology 10:1-22, which is incorporated herein by reference in its entirety.

All of the selected peptides may be tested for eliciting immune responses, an important criterion for enhancing the anti-tumor function of the immune system. For example, the strength and specificity of the immune response against cancer-targeting peptides delivered with the SAV platform may be measured. These results may be compared to previous reports for other peptide-based approaches, and any peptides that are underperforming may be identified, which guides further optimization. In some embodiments, a single identify of tumor-associated peptide is delivered using the self-assembling vaccine described herein. In some embodiments, a plurality of tumor-associated peptides are delivered. using the self-assembling vaccine. In certain embodiment, a plurality of tumor-associated peptides are delivered using multivalent self-assembling vaccine described herein. For example, a complete repertoire of peptides is delivered using the self-assembling vaccine(s) described herein, with the rationale to provide the immune system a broad collection of tumor targets. In specific embodiments, tumor associated antigen derived peptides such as peptides derived from Mesothelin or Folate Receptor Alpha can be used for making the SAVs described herein. In certain embodiments, neoantigen derived peptides such as peptides derived from Ipo13, Rpl5, or Pkp4 can be used for making the SAVs described herein. In some embodiments, peptides derived from multiple (e.g, 2, 3, 4, etc.) can be linked by a linker and used in the same SAV as a single peptide. Amino acid sequences for exemplary tumor antigens are shown below in Table 1.

TABLE 1
Representative sequences
of exemplary tumor antigens:

SEQ ID NO. 11:
PFTYEQLSIFKHKLDK
SEQ ID NO. 12:
KVSKGQKMNAQAIALVACYL
SEQ ID NO. 13:
PGFVLIWIPALLPA
SEQ ID NO. 14:
GWNWSSGHNECPVGAS
SEQ ID NO. 15:
GRCLSLLELLTVLPEEF
SEQ ID NO. 16:
TTGNKFFGALKGAVD
SEQ ID NO. 17:
NHFIIPVSTLERDRFKSHP

Included in Table 1 are polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1. Such polypeptides can have a function of the full-length polypeptide as described further herein.

In certain embodiments, the tumor antigen has an amino acid sequence that comprises at least 5, 6, 7, 8, 9, 10, 11, 12, 13, or 14 consecutive amino acids of an amino acid sequence set forth in Table 1. In some embodiments, the consecutive amino acids are identical to an amino acid sequence set forth in Table 1.

In certain embodiments, the tumor antigen has an amino acid sequence that consists essentially of at least 5, 6, 7, 8, 9, 10, 11, 12, 13, or 14 consecutive amino acids of an amino acid sequence forth in Table 1. In some embodiments, the consecutive amino acids are identical to an amino acid sequence set forth in Table 1.

In certain embodiments, the tumor antigen has an amino acid sequence that consists of at least 5, 6, 7, 8, 9, 10, 11, 12, 13, or 14 consecutive amino acids of an amino acid sequence. In some embodiments, the consecutive amino acids are identical to an amino acid sequence set forth in Table 1.

In some embodiments, the tumor antigen has an amino acid sequence that comprises 5, 6, 7, 8, 9, 10, 11, 12, 13, or 14 consecutive amino acids that are at least 70%, 75%, 80%, 85%, 90%, 91%, 92%, 93%, 94%, 95%, 96%, 97%, 98% or 99% identical to an amino acid sequence set forth in Table 1. In some embodiments, the consecutive amino acids are identical to an amino acid sequence set forth in Table 1.

In some embodiments, the tumor antigen has an amino acid sequence that consists essentially of 5, 6, 7, 8, 9, 10, 11, 12, 13, or 14 consecutive amino acids that are at least 70%, 75%, 80%, 85%, 90%, 91%, 92%, 93%, 94%, 95%, 96%, 97%, 98% or 99% identical to an amino acid sequence set forth in Table 1. In some embodiments, the consecutive amino acids are identical to an amino acid sequence set forth in Table 1.

In some embodiments, the tumor antigen has an amino acid sequence that consists of 5, 6, 7, 8, 9, 10, 11, 12, 13, or 14 consecutive amino acids that are at least 70%, 75%, 80%, 85%, 90%, 91%, 92%, 93%, 94%, 95%, 96%, 97%, 98% or 99% identical to an amino acid sequence set forth in Table 1. In some embodiments, the consecutive amino acids are identical to an amino acid sequence set forth in Table 1.

As is well-known to those skilled in the art, polypeptides having substantial sequence similarities can cause identical or very similar immune reaction in a host animal. Accordingly, in some embodiments, a derivative, equivalent, variant, fragment, or mutant of the tumor antigen protein or fragment thereof can also suitable for the methods and compositions provided herein.

In some embodiments, variations or derivatives of the tumor antigen are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well known in the art that the amino acids within the same conservative group can typically substitute for one another without substantially affecting the function of a protein. According to certain embodiments, the derivative, equivalents, variants, or mutants of the tumor antigen are polypeptides that are at least 85% homologous to a sequence of the tumor antigen protein or fragment thereof. In some embodiments, the homology is at least 90%, at least 95%, or at least 98%.

In certain embodiments, the tumor antigen may be produced by recombinant DNA techniques. For example, a nucleic acid molecule encoding the tumor antigen is cloned into an expression vector, the expression vector is introduced into a host cell and the tumor antigen is expressed in the host cell. The tumor antigen can then be isolated from the cells by an appropriate purification scheme using standard protein purification techniques.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, the tumor antigen can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

Alternative to recombinant expression, a tumor antigen can be synthesized chemically using standard peptide synthesis techniques. Chemical synthesis may be carried out using a variety of art recognized methods, including stepwise solid phase synthesis, semi-synthesis through the conformationally assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Sclmolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

Moreover, native tumor antigen can be isolated from cancer cells or tissue that harbor the tumor antigen by an appropriate purification scheme using standard protein purification techniques, for example using a tumor antigen-specific antibody. Cancer cells or tissue that harbor the tumor antigen may be isolated from a subject. The exemplary methods of isolation and purification of the tumor cell or tumor issue has been described above. In some other embodiments, the tumor antigen may be isolated from a cancer cell line that harbors the tumor antigen.

c. Biotinylated Virus or Viral Antigen

In some embodiments, biotinylated virus, or viral antigen can be administered to a subject in conjunction with a heat shock protein fusion as described herein. The subject may be afflicted with cancer that is induced by infection of a tumor-producing virus, for example, HPV, HCV, EBV, HIV, or Herpes virus. The biotinylated virus, or viral antigen may be administered to a subject in conjunction with a heat shock protein fusion as described herein to prevent and/or treat cancer. In some embodiments, the cancer is induced by infection of a tumor-producing virus, for example, HPV, EBV, HIV, or Herpes virus. In specific embodiments, the cancer is a HPV-related cancer (e.g., cervical cancer, head and neck cancer, or anal cancer).

The biotinylated virus administered in conjunction with a heat shock protein fusion as described herein may comprise a biotinylated tumor-producing virus (e.g., a HPV, HCV, EBV, HIV, or Herpes virus). In specific embodiments, the biotinylated virus is a biotinylated whole or partial inactivated tumor-producing virus (e.g., a HPV, HCV, EBV, HIV, or Herpes virus). In preferred embodiments, the biotinylated virus expresses an antigen that can induce immune response (e.g., anti-tumor immunity).

The biotinylated viral antigen administered in conjunction with a heat shock protein fusion as described herein may comprise a protein or an immunogenic fragment thereof that is derived from a tumor-producing virus (e.g., HPV, HCV, EBV, HIV, or Herpes virus). Examples of immunogenic tumor antigens that may be biotinylated are described in Stevanovic, S. et al. (2017) Science 356:200-205, which is incorporated herein by reference in its entirety. In specific embodiments, the biotinylated viral antigen is a biotinylated HPV viral antigen. The term “HPV viral antigen” refers to protein, peptide or functional equivalent fragment that is derived from HPV viral and is capable to elicit an immune response (e.g., an anti-tumor immunity). HPV viral antigens may include, but are not limited to, viral oncoproteins, E6 and E7, and immunogenic fragment thereof. E6 and E7 are the two major viral oncoproteins that may be used for developing therapeutic vaccines because they drive cellular immortalization and maintain the transformed phenotype during tumor progression. The amino acid sequence of E6 from HPV16, one of the high risk HPV types, is available to the public at the GenBank database under NP_041325.1. The amino acid sequence of E7 from HPV16 is available to the public at the GenBank database under NP_041326.1. Exemplary biotinylated viral antigens used in the compositions and methods of the present invention are listed below in Table 3 and further illustrated in the Examples.

TABLE 3
Representative amino acid
sequences for HPV viral antigens

SEQ ID NO. 3:
QLLRREVYDFAFRDLC
SEQ ID NO. 4:
GQAEPDRAHYNIVTFCCKCD
SEQ ID NO. 5:
QLLRREVYDFAFRDL
SEQ ID NO. 6:
VYDFAFRDLC
SEQ ID NO. 7:
QAEPDRAHVYNIVTFCCKCD
SEQ ID NO. 8:
GQAEPDRAHYNIVTFCCKCDSTLRLCVQSTHVDIR
SEQ ID NO. 9:
RAHVYNIVTF
SEQ ID NO. 10:
GQAEPDRAHVYNIVTFCCKCD

Included in Table 3 are polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 3. Such polypeptides can have a function of the full-length polypeptide as described further herein.

### Heat Shock Protein Fusions

A “heat shock protein” is encoded by a “heat shock gene” or a stress gene, and refers a gene that is activated or otherwise detectably upregulated due to the contact or exposure of an organism (containing the gene) to a stressor, such as heat shock, hypoxia, glucose deprivation, heavy metal salts, inhibitors of energy metabolism and electron transport, and protein denaturants, or to certain benzoquinone ansamycins. Nover, L., Heat Shock Response, CRC Press, Inc., Boca Raton, Fla. (1991). “Heat shock protein” also includes homologous proteins encoded by genes within known stress gene families, even though such homologous genes are not themselves induced by a stressor.

A “heat shock protein fusion” refers to a heat shock protein linked to a biotin-binding protein. For example, a heat shock protein may be C- or N-terminally joined to a biotin-binding protein to generate a heat shock protein fusion. When administered in conjunction with a biotinylated component (e.g., a tumor cell or a tumor antigen) provided herein, a heat shock protein fusion is capable of stimulating or enhancing humoral and/or cellular immune responses, including CD8 cytotoxic T cell (CTL) responses, to an antigen of interest.

For example, but not by way of limitation, heat shock proteins which may be used according to the invention include BiP (also referred to as grp78), Hsp10, Hsp20-30, Hsp60 hsp70, hsc70, gp96 (grp94), hsp60, hsp40, and Hsp100-200, Hsp100, Hsp90, and members of the families thereof. Especially preferred heat shock proteins are BiP, gp96, and hsp70, as exemplified below. A particular group of heat shock proteins includes Hsp90, Hsp70, Hsp60, Hsp20-30, further preferably Hsp70 and Hsp60. Most preferred is a member of the hsp70 family.

Hsp10 examples include GroES and Cpn10. Hsp10 is typically found in E. coli and in mitochondria and chloroplasts of eukaryotic cells. Hsp10 forms a seven-membered ring that associates with Hsp60 oligomers. Hsp10 is also involved in protein folding.

Hsp60 examples include Hsp65 from mycobacteria. Bacterial Hsp60 is also commonly known as GroEL, such as the GroEL from E. coli. Hsp60 forms large homooligomeric complexes, and appears to play a key role in protein folding. Hsp60 homologues are present in eukaryotic mitochondria and chloroplasts.

Hsp70 examples include Hsp72 and Hsc73 from mammalian cells, DnaK from bacteria, particularly mycobacteria such as Mycobacterium leprae, Mycobacterium tuberculosis (MTb), and Mycobacterium bovis (such as Bacille-Calmette Guerin; referred to herein as Hsp71), DnaK from Escherichia coli, yeast, and other prokaryotes, and BiP and Grp78. Hsp70 is capable of specifically binding ATP as well as unfolded proteins, thereby participating in protein folding and unfolding as well as in the assembly and disassembly of protein complexes. In a preferred embodiment, the heat shock protein is or is derived from MTb HSP 70. The full-length protein sequences of Mycobacterium tuberculosis HSP70 and Mycobacterium bovis HSP70 are depicted in Table 2 as SEQ ID NOs: 1 and 2, respectively. A heat shock protein fusion to be used in conjunction with the methods described herein may comprise a sequence that is at least 80%, 85%, 90%, 95%, or 99% identical to SEQ ID NO: 1 or 2.

TABLE 2
Chaperone protein dnaK (Heat shock protein 70) from
Mycobacterium tuberculosis (P0A5B9, GI: 61222666)
SEQ ID NO: 1

| | |
|---|---|
| 1 | maravgidlg ttnsvvsvle ggdpvvvans egsrttpsiv afarngevlv gqpaknqavt |
| 61 | nvdrtvrsvk rhmgsdwsie idgkkytape isarilmklk rdaeaylged itdavittpa |
| 121 | yfndaqrqat kdagqiagln vlrivnepta aalaygldkg ekeqrilvfd lgggtfdvsl |
| 181 | leigegvvev ratsgdnhlg gddwdqrvvd wlvdkfkgts gidltkdkma mqrlreaaek |
| 241 | akielsssqs tsinlpyitv dadknplfld eqltraefqr itqdlldrtr kpfqsviadt |
| 301 | gisvseidhv vlvggstrmp avtdlvkelt ggkepnkgvn pdevvavgaa lqagvlkgev |
| 361 | kdvllldvtp lslgietkgg vmtrliernt tiptkrsetf ttaddnqpsv qiqvyqgere |
| 421 | iaahnkllgs feltgippap rgipqievtf didangivhv takdkgtgke ntiriqegsg |
| 481 | lskedidrmi kdaeahaeed rkrreeadvr nqaetlvyqt ekfvkeqrea eggskvpedt |
| 541 | lnkvdaavae akaalggsdi saiksamekl gqesqalgqa iyeaaqaasq atgaahpgge |
| 601 | pggahpgsad dvvdaevvdd greak |

Chaperone protein dnaK (Heat shock protein 70) from
Mycobacterium bovis (NP_854021.1 GI: 31791528)
SEQ ID NO: 2

| | |
|---|---|
| 1 | maravgidlg ttnsvvsvle ggdpvvvans egsrttpsiv afarngevlv gqpaknqavt |
| 61 | nvdrtvrsvk rhmgsdwsie idgkkytape isarilmklk rdaeaylged itdavittpa |
| 121 | yfndaqrqat kdagqiagln vlrivnepta aalaygldkg ekeqrilvfd lgggtfdvsl |
| 181 | leigegvvev ratsgdnhlg gddwdqrvvd wlvdkfkgts gidltkdkma mqrlreaaek |
| 241 | akielsssqs tsinlpyitv dadknplfld eqltraefqr itqdlldrtr kpfqsviadt |
| 301 | gisvseidhv vlvggstrmp avtdlvkelt ggkepnkgvn pdevvavgaa lqagvlkgev |
| 361 | kdvllldvtp lslgietkgg vmtrliernt tiptkrsetf ttaddnqpsv qiqvyqgere |
| 421 | iaahnkllgs feltgippap rgipqievtf didangivhv takdkgtgke ntiriqegsg |
| 481 | lskedidrmi kdaeahaeed rkrreeadvr nqaetlvyqt ekfvkegrea eggskvpedt |
| 541 | lnkvdaavae akaalggsdi saiksamekl gqesqalgqa iyeaaqaasq atgaahpgge |
| 601 | pggahpgsad dvvdaevvdd greak |

Included in Table 2 are polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 2. Such polypeptides can have a function of the full-length polypeptide as described further herein.

Hsp90 examples include HtpG in E. coli, Hsp83 and Hsc83 yeast, and Hsp90 alpha, Hsp90 beta and Grp94 in humans. Hsp90 binds groups of proteins, which proteins are typically cellular regulatory molecules such as steroid hormone receptors (e.g., glucocorticoid, estrogen, progesterone, and testosterone receptors), transcription factors and protein kinases that play a role in signal transduction mechanisms. Hsp90 proteins also participate in the formation of large, abundant protein complexes that include other heat shock proteins.

Hsp100 examples include mammalian Hsp 110, yeast Hsp104, ClpA, ClpB, ClpC, ClpX and ClpY. Yeast Hsp104 and E. coli ClpA, form hexameric and E. coli ClpB, tetrameric particles whose assembly appears to require adenine nucleotide binding. Clp protease provides a 750 kDa heterooligomer composed of ClpP (a proteolytic subunit) and of ClpA. ClpB-Y are structurally related to ClpA, although unlike ClpA they do not appear to complex with ClpP.

Hsp100-200 examples include Grp170 (for glucose-regulated protein). Grp170 resides in the lumen of the ER, in the pre-golgi compartment, and may play a role in immunoglobulin folding and assembly.

Naturally occurring or recombinantly derived mutants of heat shock proteins may be used according to the invention. For example, but not by way of limitation, the present invention provides for the use of heat shock proteins mutated so as to facilitate their secretion from the cell (for example having mutation or deletion of an element which facilitates endoplasmic reticulum recapture, such as KDEL or its homologues; such mutants are described in PCT Application No. PCT/US96/13233 (WO 97/06685), which is incorporated herein by reference).

In particular embodiments, the heat shock proteins of the present invention are obtained from enterobacteria, mycobacteria (particularly M. leprae, M. tuberculosis, M. vaccae, M. smegmatis and M. bovis), E. coli, yeast, Drosophila, vertebrates, avians, chickens, mammals, rats, mice, primates, or humans.

The pharmaceutical compositions provided herein may have individual amino acid residues that are modified by oxidation or reduction. Furthermore, various substitutions, deletions, or additions may be made to the amino acid or nucleic acid sequences, the net effect of which is to retain or further enhance the increased biological activity of the heat shock protein. Due to code degeneracy, for example, there may be considerable variation in nucleotide sequences encoding the same amino acid sequence. The term “heat shock protein” is intended to encompass fragments of heat shock proteins obtained from heat shock proteins, provided such fragments include the epitopes involved with enhancing the immune response to an antigen of interest. Fragments of heat shock proteins may be obtained using proteinases, or by recombinant methods, such as the expression of only part of a stress protein-encoding nucleotide sequence (either alone or fused with another protein-encoding nucleic acid sequence). The heat shock proteins may include mutations introduced at particular loci by a variety of known techniques to enhance its effect on the immune system. See, e.g., Sambrook et al., Molecular Cloning: A Laboratory Manual, 2d Ed., Cold Spring Harbor Laboratory Press (1989); Drinkwater and Klinedinst Proc. Natl. Acad. Sci. USA 83:3402-3406 (1986); Liao and Wise, Gene 88:107-111 (1990); Horwitz et al., Genome 3:112-117 (1989).

In particular embodiments, e.g., in heat shock protein fusions involving chemical conjugates between a heat shock protein and a biotin-binding protein, the heat shock proteins used in the present invention are isolated heat shock proteins, which means that the heat shock proteins have been selected and separated from the host cell in which they were produced. In some embodiments where the heat shock is expressed recombinantly as a fusion of a heat shock protein fused to a biotin-binding protein, the heat shock protein fusions used in the present invention are isolated heat shock protein fusions, which means that the heat shock protein fusions have been selected and separated from the host cell in which they were produced. Such isolation can be carried out as described herein and using routine methods of protein isolation known in the art. Maniatis et al., Molecular Cloning, A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press (1989); Deutscher, M., Guide to Protein Purification Methods Enzymology, vol. 182, Academic Press, Inc., San Diego, Calif. (1990). Examples of methods for producing a fusion of a heat shock protein fused to a biotin-binding protein are further described in the PCT Publication No. WO 2009/129502, the entire contents of which are incorporated herein by reference.

### Self-Assembling Vaccines

Multiple biotinylated components (e.g., tumor cells or tumor antigens) may be administered in conjunction with a heat shock protein fusion as further described. In this way, multivalent pharmaceutical compositions may be generated and administered to a subject. The generation of multivalent pharmaceutical compositions allow for the production of “supercharged,” or more potent vaccines and therapeutics.

Wherein the pharmaceutical composition is multivalent, the biotinylated components (e.g., tumor cells or tumor antigens) to be administered may be any combination of biotinylated components (e.g., tumor cells or tumor antigens) described herein. For example, biotinylated components (e.g., tumor cells or tumor antigens) of the same or different identities may be administered in conjunction with a heat shock protein fusion as provided herein, provided that the biotin-binding protein, and in turn the heat shock protein fusion, is multivalent, or capable of binding multiple biotinylated components (e.g., tumor cells or tumor antigens). As an example, the wild-type biotin-binding protein avidin has four biotin-binding sites and is therefore capable of binding four biotinylated components (e.g., tumor cells or tumor antigens). In this example, the four sites are to be bound by four biotinylated components (e.g., tumor cells or tumor antigens), and the biotin-binding components (e.g., tumor cells or tumor antigens) may be mixed and matched based on identity in any possible permutation of one, two, three, or four identical biotinylated components (e.g., tumor cells or tumor antigens) described herein. Four identical biotinylated components (e.g., tumor cells or tumor antigens) may be bound to the four biotin-binding sites.

Therefore, an effective amount of a biotinylated tumor cell or tumor antigen with a first identity may be administered to a subject in conjunction with a heat shock protein fused to a biotin-binding protein, sufficient to form a pharmaceutical composition comprising four parts biotinylated tumor cell or tumor antigen of a first identity and one part heat shock protein fused to a biotin-binding protein. Alternatively, an effective amount of biotinylated tumor cells or tumor antigens with a first and second identity may be may be administered to a subject in conjunction with a heat shock protein fused to a biotin-binding protein, sufficient to form a pharmaceutical composition comprising three parts biotinylated tumor cells or tumor antigens of a first identity, one part biotinylated tumor cell or tumor antigen of a second identity, and one part heat shock protein fusion. In another embodiment, an effective amount of biotinylated tumor cells or tumor antigens with a first and second identity may be administered to a subject in conjunction with a heat shock protein fused to a biotin-binding protein, sufficient to form a pharmaceutical composition comprising two parts biotinylated tumor cells or tumor antigens of a first identity, two parts biotinylated tumor cells or tumor antigens of a second identity, and one part heat shock protein fusion.

Wherein the self-assembling pharmaceutical composition is divalent, an effective amount of biotinylated tumor cell or tumor antigen of a first identity may be administered to a subject in conjunction with a heat shock protein fused to a biotin-binding protein, sufficient to form a pharmaceutical composition comprising two parts of biotinylated tumor cell or tumor antigen of a first identity and one part heat shock protein fusion. Alternatively, an effective amount of biotinylated tumor cells or tumor antigens with a first and second identity may be may be administered to a subject in conjunction with a heat shock protein fused to a biotin-binding protein, sufficient to form a pharmaceutical composition comprising one part biotinylated tumor cell or tumor antigen of a first identity, one part biotinylated tumor cell or tumor antigen of a second identity, and one part heat shock protein fusion.

The multivalent pharmaceutical composition may include a costimulatory molecule, or a blocking group (i.e., biotin alone or biotin conjugated to a non-functional molecule). Examples of costimulatory molecules that may be administered in conjunction with the present invention include B7 molecules, including B7-1 (CD80) and B7-2 (CD86), CD28, CD58, LFA-3, CD40, B7-H3, CD137 (4-1BB), and interleukins (e.g., IL-1, IL-2, or IL-12). As an example, one part biotinylated component comprising a costimulatory molecule may be administered in conjunction with i) three parts of another biotinylated component comprising a tumor cell or tumor antigen; and ii) one part heat shock protein fused to a biotin-binding protein. In another example, two parts biotinylated component comprising a costimulatory molecule may be administered in conjunction with i) two parts of another biotinylated component comprising a tumor cell or tumor antigen; and ii) one part heat shock protein fused to a biotin-binding protein. In another example, three parts biotinylated component comprising a costimulatory molecule may be administered in conjunction with i) one part of another biotinylated component comprising a tumor cell or tumor antigen; and ii) one part heat shock protein fused to a biotin-binding protein.

A pH-sensitive mutant of avidin, streptavidin, or neutravidin, for example, may be employed to control the noncovalent interaction of avidin-, streptavidin-, or neutravidin- to biotin, and thereby achieve the desired stoichiometry of heat shock protein fusion with the various permutations and combinations of biotinylated tumor cells or tumor antigens, as described herein. The choice of wild-type or a particular mutant form of biotin-binding protein such as avidin may be employed to control the desired valency of the pharmaceutical composition (e.g., monomeric, dimeric, or tetrameric form of avidin). Monovalent or divalent vaccines may be similarly produced by employing heat shock fusion proteins comprising other avidin, streptavidin, or neutravidin mutant proteins that bind biotin but in a monovalent or divalent fashion. An example of an avidin mutant is described in the Exemplification section below. An example of a pH-sensitive point mutant of Avidin which confers pH-adjustable biotin binding is Y33H. Another mutant has substitutions of histidine for Met96, Val115, and Ile117, optionally with histidine replacement at Trp110. Such approaches for controlling biotin-streptavidin binding are described in Laitinen, 0. H. (2007), “Brave New (Strept)avidins in Biotechnology,” Trends in Biotechnology 25 (6): 269-277 and Nordlund, H. R. (2003), “Introduction of histidine residues into avidin subunit interfaces allows pH-dependent regulation of quaternary structure and biotin binding,” FEBS Letters 555: 449-454, the contents of both of which are incorporated herein by reference.

### Methods of Producing the Self-Assembling Vaccines

In one embodiment of the present invention, compositions are comprised of two moieties: a heat shock protein fused to a biotin-binding protein and a biotinylated component (e.g., tumor cell or tumor antigen), which targets the immune response to the antigen to which the immune response is desired. The present invention provides for fast, easy production of large amounts pharmaceutical composition (e.g., vaccine) because the production of biotinylated antigens or antibodies is well known and rapid, which, in turn, allows for an increased capacity for vaccine production. Because a heat shock protein fusion of a single identity may be administered in conjunction with any of a number of various biotinylated components (e.g., tumor cell or tumor antigen) as described herein, the heat shock fusion protein need not be synthesized de novo each time a new target antigen of interest is identified. Therefore, such methods of production are particularly rapid once the heat shock protein fusion to be administered is established and has been produced.

Provided are methods for making the heat shock protein fused to a biotin-binding protein. The heat shock protein may be prepared, using standard techniques, from natural sources, for example as described in Flynn et al., Science 245:385-390 (1989), or using recombinant techniques such as expression of a heat shock encoding gene construct in a suitable host cell such as a bacterial, yeast or mammalian cell. A fusion protein including the heat shock protein and biotin-binding protein can be produced by recombinant means. For example, a nucleic acid encoding the heat shock protein can be joined to either end of a nucleic acid sequence encoding the biotin-binding protein such that the two protein-coding sequences are sharing a common translational reading frame and can be expressed as a fusion protein including the biotin-binding protein and the heat shock protein. The combined sequence is inserted into a suitable vector chosen based on the expression features desired and the nature of the host cell. In the examples provided hereinafter, the nucleic acid sequences are assembled in a vector suitable for protein expression in the bacterium E. coli. Following expression in the chosen host cell, the fusion protein can be purified by routine biochemical separation techniques or by immunoaffinity methods using an antibody to one or the other part of the fusion protein. Alternatively, the selected vector can add a tag to the fusion protein sequence, e.g., an oligohistidine tag as described in the examples presented hereinafter, permitting expression of a tagged fusion protein that can be purified by affinity methods using an antibody or other material having an appropriately high affinity for the tag. Sambrook et al., Molecular Cloning: A Laboratory Manual, 2d Ed., Cold Spring Harbor Laboratory Press (1989); Deutscher, M. Guide to Protein Purification Methods Enzymology, vol. 182. Academic Press, Inc. San Diego, Calif. (1990). If a vector suitable for expression in mammalian cells is used. e.g., one of the vectors discussed below, the heat shock protein fusion can be expressed and purified from mammalian cells. Alternatively, the mammalian expression vector (including fusion protein-coding sequences) can be administered to a subject to direct expression of heat shock protein fusion protein in the subject's cells. A nucleic acid encoding a heat shock protein can also be produced chemically and then inserted into a suitable vector for fusion protein production and purification or administration to a subject. Finally, a fusion protein can also be prepared chemically.

Techniques for making fusion genes are well known in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992). Accordingly, provided is an isolated nucleic acid comprising a fusion gene of a gene encoding a heat shock protein fused to a gene encoding a biotin-binding protein.

The nucleic acid may be provided in a vector comprising a nucleotide sequence encoding the heat shock protein fusion, and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered. Such vectors may be administered in any biologically effective carrier, e.g., any formulation or composition capable of effectively transfecting cells either ex vivo or in vivo with genetic material encoding a chimeric polypeptide. Approaches include insertion of the nucleic acid in viral vectors including recombinant retroviruses, adenoviruses, adeno-associated viruses, human immunodeficiency viruses, and herpes simplex viruses-1, or recombinant bacterial or eukaryotic plasmids. Viral vectors may be used to transfect cells directly; plasmid DNA may be delivered alone with the help of, for example, cationic liposomes (lipofectin) or derivatized (e.g., antibody conjugated), polylysine conjugates, gramicidin S, artificial viral envelopes or other such intracellular carriers. Nucleic acids may also be directly injected. Alternatively, calcium phosphate precipitation may be carried out to facilitate entry of a nucleic acid into a cell.

The subject nucleic acids may be used to cause expression and over-expression of a heat shock protein fusion protein in cells propagated in culture, e.g. to produce fusion proteins.

Provided also is a host cell transfected with a recombinant gene in order to express the heat shock protein fusion. The host cell may be any prokaryotic or eukaryotic cell. For example, a heat shock protein fusion may be expressed in bacterial cells, such as E. coli, insect cells (baculovirus), yeast, insect, plant, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the fusion polypeptide will be known to those in the art.

A cell culture includes host cells, media and other byproducts. Suitable media for cell culture are well known in the art. A fusion polypeptide may be secreted and isolated from a mixture of cells and medium comprising the polypeptide. Alternatively, a fusion polypeptide may be retained cytoplasmically and the cells harvested, lysed and the protein isolated. A fusion polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a fusion.

Thus, a nucleotide sequence encoding all or part of the heat shock protein fusion may be used to produce a recombinant form of a protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant fusion polypeptides by microbial means or tissue-culture technology in accord with the subject invention.

Expression vehicles for production of a recombinant protein include plasmids and other vectors. For instance, suitable vectors for the expression of a fusion polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

In another embodiment, the nucleic acid encoding the heat protein fusion polypeptide is operably linked to a bacterial promoter, e.g., the anaerobic E. coli, NirB promoter or the E. coli lipoprotein llp promoter, described, e.g., in Inouye et al. (1985) Nucl. Acids Res. 13:3101; Salmonella pagC promoter (Miller et al., supra), Shigella ent promoter (Schmitt and Payne, J. Bacteriol. 173:816 (1991)), the tet promoter on Tn10 (Miller et al., supra), or the ctx promoter of Vibrio cholera. Any other promoter can be used. The bacterial promoter can be a constitutive promoter or an inducible promoter. An exemplary inducible promoter is a promoter which is inducible by iron or in iron-limiting conditions. In fact, some bacteria, e.g., intracellular organisms, are believed to encounter iron-limiting conditions in the host cytoplasm. Examples of iron-regulated promoters of FepA and TonB are known in the art and are described, e.g., in the following references: Headley, V. et al. (1997) Infection & Immunity 65:818; Ochsner, U. A. et al. (1995) Journal of Bacteriology 177:7194; Hunt, M. D. et al. (1994) Journal of Bacteriology 176:3944; Svinarich, D. M. and S. Palchaudhuri. (1992) Journal of Diarrhoeal Diseases Research 10:139; Prince, R. W. et al. (1991) Molecular Microbiology 5:2823; Goldberg, M. B. et al. (1990) Journal of Bacteriology 172:6863; de Lorenzo, V. et al. (1987) Journal of Bacteriology 169:2624; and Hantke, K. (1981) Molecular & General Genetics 182:288.

A plasmid preferably comprises sequences required for appropriate transcription of the nucleic acid in bacteria, e.g., a transcription termination signal. The vector can further comprise sequences encoding factors allowing for the selection of bacteria comprising the nucleic acid of interest, e.g., gene encoding a protein providing resistance to an antibiotic, sequences required for the amplification of the nucleic acid, e.g., a bacterial origin of replication.

In another embodiment, a signal peptide sequence is added to the construct, such that the fusion polypeptide is secreted from cells. Such signal peptides are well known in the art.

In one embodiment, the powerful phage T5 promoter, that is recognized by E. coli RNA polymerase is used together with a lac operator repression module to provide tightly regulated, high level expression or recombinant proteins in E. coli. In this system, protein expression is blocked in the presence of high levels of lac repressor.

In one embodiment, the DNA is operably linked to a first promoter and the bacterium further comprises a second DNA encoding a first polymerase which is capable of mediating transcription from the first promoter, wherein the DNA encoding the first polymerase is operably linked to a second promoter. In a preferred embodiment, the second promoter is a bacterial promoter, such as those delineated above. In an even more preferred embodiment, the polymerase is a bacteriophage polymerase, e.g., SP6, T3, or T7 polymerase and the first promoter is a bacteriophage promoter, e.g., an SP6, T3, or T7 promoter, respectively. Plasmids comprising bacteriophage promoters and plasmids encoding bacteriophage polymerases can be obtained commercially, e.g., from Promega Corp. (Madison, Wis.) and InVitrogen (San Diego, Calif.), or can be obtained directly from the bacteriophage using standard recombinant DNA techniques (J. Sambrook, E. Fritsch, T. Maniatis, Molecular Cloning: A Laboratory Manual, Cold Spring Laboratory Press, 1989). Bacteriophage polymerases and promoters are further described, e.g., in the following references: Sagawa, H. et al. (1996) Gene 168:37; Cheng, X. et al. (1994) PNAS USA 91:4034; Dubendorff, J. W. and F. W. Studier (1991) Journal of Molecular Biology 219:45; Bujarski, J. J. and P. Kaesberg (1987) Nucleic Acids Research 15:1337; and Studier, F. W. et al. (1990) Methods in Enzymology 185:60). Such plasmids can further be modified according to the specific embodiment of the heat shock protein fusion to be expressed.

In another embodiment, the bacterium further comprises a DNA encoding a second polymerase which is capable of mediating transcription from the second promoter, wherein the DNA encoding the second polymerase is operably linked to a third promoter. The third promoter may be a bacterial promoter. However, more than two different polymerases and promoters could be introduced in a bacterium to obtain high levels of transcription. The use of one or more polymerases for mediating transcription in the bacterium can provide a significant increase in the amount of polypeptide in the bacterium relative to a bacterium in which the DNA is directly under the control of a bacterial promoter. The selection of the system to adopt will vary depending on the specific use, e.g., on the amount of protein that one desires to produce.

Generally, a nucleic acid encoding a fusion protein is introduced into a host cell, such as by transfection, and the host cell is cultured under conditions allowing expression of the fusion protein. Methods of introducing nucleic acids into prokaryotic and eukaryotic cells are well known in the art. Suitable media for mammalian and prokaryotic host cell culture are well known in the art. Generally, the nucleic acid encoding the subject fusion protein is under the control of an inducible promoter, which is induced once the host cells comprising the nucleic acid have divided a certain number of times. For example, where a nucleic acid is under the control of a beta-galactose operator and repressor, isopropyl beta-D-thiogalactopyranoside (IPTG) is added to the culture when the bacterial host cells have attained a density of about OD₆₀₀ 0.45-0.60. The culture is then grown for some more time to give the host cell the time to synthesize the protein. Cultures are then typically frozen and may be stored frozen for some time, prior to isolation and purification of the protein.

When using a prokaryotic host cell, the host cell may include a plasmid which expresses an internal T7 lysozyme, e.g., expressed from plasmid pLysSL (see Examples). Lysis of such host cells liberates the lysozyme which then degrades the bacterial membrane.

Other sequences that may be included in a vector for expression in bacterial or other prokaryotic cells include a synthetic ribosomal binding site; strong transcriptional terminators, e.g., t₀ from phage lambda and t₄ from the rrnB operon in E. coli, to prevent read through transcription and ensure stability of the expressed protein; an origin of replication, e.g., ColE1; and beta-lactamase gene, conferring ampicillin resistance.

Other host cells include prokaryotic host cells. Even more preferred host cells are bacteria, e.g., E. coli. Other bacteria that can be used include Shigella spp., Salmonella spp., Listeria spp., Rickettsia spp., Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., and Erysipelothrix spp. Most of these bacteria can be obtained from the American Type Culture Collection (ATCC; 10801 University Blvd., Manassas, Va. 20110-2209).

A number of vectors exist for the expression of recombinant proteins in yeast. For instance, YEP24, YIPS, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal comprising pBlueBac III).

In another variation, protein production may be achieved using in vitro translation systems. In vitro translation systems are, generally, a translation system which is a cell-free extract comprising at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. An RNA nucleotide for in vitro translation may be produced using methods known in the art. In vitro transcription and translation may be coupled in a one-pot reaction to produce proteins from one or more isolated DNAs.

When expression of a carboxy terminal fragment of a protein is desired, i.e., a truncation mutant, it may be necessary to add a start codon (ATG) to the oligonucleotide fragment comprising the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant proteins in a host which produces MAP (e.g., E. coli or CM89 or S. cerevisiae), or in vitro by use of purified MAP (e.g., procedure of Miller et al.).

In cases where plant expression vectors are used, the expression a heat shock protein fusion may be driven by any of a number of promoters. For example, viral promoters such as the 35S RNA and 19S RNA promoters of CaMV (Brisson et al., 1984, Nature, 310:511-514), or the coat protein promoter of TMV (Takamatsu et al., 1987, EMBO J., 6:307-311) may be used; alternatively, plant promoters such as the small subunit of RUBISCO (Coruzzi et al., 1994, EMBO J., 3:1671-1680; Broglie et al., 1984, Science, 224:838-843); or heat shock promoters, e.g., soybean Hsp 17.5-E or Hsp 17.3-B (Gurley et al., 1986, Mol. Cell. Biol., 6:559-565) may be used. These constructs can be introduced into plant cells using Ti plasmids, Ri plasmids, plant virus vectors; direct DNA transformation; microinjection, electroporation, etc. For reviews of such techniques see, for example, Weissbach & Weissbach, 1988, Methods for Plant Molecular Biology, Academic Press, New York, Section VIII, pp. 421-463; and Grierson & Corey, 1988, Plant Molecular Biology, 2d Ed., Blackie, London, Ch. 7-9.

An alternative expression system which can be used to express a protein tag or fusion protein comprising a protein tag is an insect system. In one such system, Autographa californica nuclear polyhedrosis virus (AcNPV) is used as a vector to express foreign genes. The virus grows in Spodoptera frugiperda cells. The PGHS-2 sequence may be cloned into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983, J. Virol., 46:584, Smith, U.S. Pat. No. 4,215,051).

In a specific embodiment of an insect system, the DNA encoding the heat shock protein fusion protein is cloned into the pBlueBacIII recombinant transfer vector (Invitrogen, San Diego, Calif.) downstream of the polyhedrin promoter and transfected into Sf9 insect cells (derived from Spodoptera frugiperda ovarian cells, available from Invitrogen, San Diego, Calif.) to generate recombinant virus. After plaque purification of the recombinant virus high-titer viral stocks are prepared that in turn would be used to infect Sf9 or High Five™ (BTI-TN-5B1-4 cells derived from Trichoplusia ni egg cell homogenates; available from Invitrogen, San Diego, Calif.) insect cells, to produce large quantities of appropriately post-translationally modified subject protein.

In other embodiments, the heat shock protein fusion and biotin-binding protein are produced separately and then linked, e.g. covalently linked, to each other. For example, a heat shock protein fusion and biotin-binding protein are produced separately in vitro, purified, and mixed together under conditions under which the tag will be able to be linked to the protein of interest. For example, the heat shock protein and/or the biotin-binding protein can be obtained (isolated) from a source in which it is known to occur, can be produced and harvested from cell cultures, can be produced by cloning and expressing a gene encoding the desired heat shock protein fusion, or can be synthesized chemically. Furthermore, a nucleic acid sequence encoding the desired heat shock protein fusion can be synthesized chemically. Such mixtures of conjugated proteins may have properties different from single fusion proteins.

Linkers (also known as “linker molecules” or “cross-linkers”) may be used to conjugate a heat shock protein and biotin-binding protein. Linkers include chemicals able to react with a defined chemical group of several, usually two, molecules and thus conjugate them. The majority of known cross-linkers react with amine, carboxyl, and sulfhydryl groups. The choice of target chemical group is crucial if the group may be involved in the biological activity of the proteins to be conjugated. For example, maleimides, which react with sulfhydryl groups, may inactivate Cys-comprising proteins that require the Cys to bind to a target. Linkers may be homofunctional (comprising reactive groups of the same type), heterofunctional (comprising different reactive groups), or photoreactive (comprising groups that become reactive on illumination).

Linker molecules may be responsible for different properties of the conjugated compositions. The length of the linker should be considered in light of molecular flexibility during the conjugation step, and the availability of the conjugated molecule for its target (cell surface molecules and the like.) Longer linkers may thus improve the biological activity of the compositions of the present invention, as well as the ease of preparation of them. The geometry of the linker may be used to orient a molecule for optimal reaction with a target. A linker with flexible geometry may allow the cross-linked proteins to conformationally adapt as they bind other proteins. The nature of the linker may be altered for other various purposes. For example, the aryl-structure of MBuS was found less immunogenic than the aromatic spacer of MBS. Furthermore, the hydrophobicity and functionality of the linker molecules may be controlled by the physical properties of component molecules. For example, the hydrophobicity of a polymeric linker may be controlled by the order of monomeric units along the polymer, e.g. a block polymer in which there is a block of hydrophobic monomers interspersed with a block of hydrophilic monomers.

The chemistry of preparing and utilizing a wide variety of molecular linkers is well-known in the art and many pre-made linkers for use in conjugating molecules are commercially available from vendors such as Pierce Chemical Co., Roche Molecular Biochemicals, United States Biological, and the like.

The prepared and/or isolated heat shock protein fused to a biotin-binding protein is to be administered to a subject in conjunction with the desired biotinylated components, sufficient to form a non-covalent association of the biotin moiety with the biotin-binding protein. The heat shock protein fusion and the biotinylated component or components (e.g., tumor cells or tumor antigens) may be administered simultaneously or sequentially. If administered simultaneously, the heat shock protein fusion and the biotinylated component or components (e.g., tumor cells or tumor antigens) may be administered as a mixture or as a noncovalent complex. If administered as a noncovalent complex, a heat shock protein fused to a biotin-binding protein may be noncovalently bound to the desired biotinylated components (e.g., tumor cells or tumor antigens) either in vitro or in vivo once prepared and/or isolated.

The noncovalent complex may be produced by contacting the heat shock protein fused to a biotin-binding protein with the biotinylated components (e.g., tumor cells or tumor antigens), under conditions sufficient to promote the binding of the biotin-binding protein with biotin, which conditions are known in the art.

Genes for various heat shock proteins have been cloned and sequenced, and which may be used to obtain a heat shock protein fusion, including, but not limited to, gp96 (human: Genebank Accession No. X15187; Maki et al., Proc. Natl. Acad. Sci. U.S.A. 87:5658-5562 (1990); mouse: Genebank Accession No. M16370; Srivastava et al., Proc. Natl. Acad. Sci. U.S.A. 84:3807-3811 (1987)), BiP (mouse: Genebank Accession No. U16277; Haas et al., Proc. Natl. Acad. Sci. U.S.A. 85:2250-2254 (1988); human: Genebank Accession No. M19645; Ting et al., DNA 7:275-286 (1988)), hsp70 (mouse: Genebank Accession No. M35021; Hunt et al., Gene 87:199-204 (1990); human: Genebank Accession No. M24743; Hunt et al, Proc. Natl. Acad. Sci. U.S.A. 82:6455-6489 (1995)), and hsp40 (human: Genebank Accession No. D49547; Ohtsuka K., Biochem. Biophys. Res. Commun. 197:235-240 (1993)).

The heat shock protein fused to a biotin-binding protein may be non-covalently bound to the biotinylated component (e.g., tumor cell or tumor antigen).

The tumor cell or tumor antigen to be administered in conjunction with the heat shock protein may be conjugated to biotin by means such as is known in the art. Prior to conjugation to biotin, the tumor cell or tumor antigen may be produced and/or isolated using methods known in the art. Recombinant techniques may be employed in much the same way as described herein for the heat shock protein fusion. Once the tumor cell or tumor antigen is produced and/or isolated, a biotin molecule or molecules may be conjugated directly to a tumor cell or tumor antigen. Biotin may also be conjugated indirectly through a linker to said tumor cell or tumor antigen Biotin is to be conjugated to a region that sterically allows for the interaction of biotin with the biotin-binding protein. Biotinylation kits and reagents may be purchased from Pierce (Rockford, Ill.) and used to generate the biotinylated components described herein.

The sequences of many different antigens can be cloned and characterized by DNA sequence analysis and included in the compositions provided herein. Bacterial vectors containing complete or partial cellular or viral genomes or antigens may be obtained from various sources including, for example, the American Tissue Culture Collection (ATCC). Additional antigens which may be used can be isolated and typed by the methods previously established for this purpose, which methods are well known in the art.

### Immunotherapy

In some aspects, the self-assembling vaccine described herein can be administered in combination with an immunotherapy.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose Immunotherapies that are designed to elicit or amplify an immune response are referred to as “activation immunotherapies” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen) Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, the immunotherapy described herein comprises at least one immunogenic chemotherapies. The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013), Annu. Rev. Immunol., 31:51-72). Specific representative examples of consensus immunogenic chemotherapies include 5′-fluorouracil, anthracyclines, such as doxorubicin, and platinum drugs, such as oxaliplatin, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily Other IgC domains fall within other sets. IgV domains also share sequence patterns and are called V set domains. IgV domains are longer than IgC domains and contain an additional pair of 13 strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1 S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. In addition, nucleic acid and polypeptide sequences of PD-L1 orthologs in organisms other than humans are well-known and include, for example, mouse PD-L1 (NM_021893.3 and NP_068693.1), rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two 13 sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, mouse PD-L2 (NM_021396.2 and NP_067371.1), rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra-or intercellular signaling, 3) modulate activation of immune cells, e.g., T lymphocytes, and 4) modulate the immune response of an organism, e.g., a mouse or human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

In preferred embodiments, the immunotherapy used in the compositions and methods of the present invention is an agent that inhibits PD1 or PD-L1. Such agents include, but are not limited to, small molecule inhibitors, CRISPR guide RNAs (gRNA), RNA interfering agents, antisense oligonucleotides, peptides or peptidomimetic inhibitors, aptamers, antibodies, or intrabodies. In specific embodiments, the agent that inhibits PD1 or PD-L1 is a PD1 or PD-L1 blocking antibody. Exemplary anti-PD-1 antibodies can be used in the present invention include, but are not limited to, Keytruda (Merck, Inc.).

In some embodiments, the immunotherapy used in the compositions and methods of the present invention is an immune modulatory agent. Such agents include, but are not limited to, CXCR4/CXCR7 antagonists (e.g., AMD3100), Jak/stat inhibitors (e.g., Ruxolitinib), and near infrared laser immunomodulation of skin associated immune cells. Examples of near infrared laser immunomodulation of skin-associated immune cells are described in Kimizuka, Y. et al. J. Immun 2018, 201(12) 3587-3603 and Gelfand, J. et al. FASEB J. 2019, 33(2), 3074-3081, which are incorporated by reference in their entireties.

### Pharmaceutical Compositions and Administration

In some embodiments, a pharmaceutical composition provided herein comprises a heat shock protein fused to a biotin-binding protein, wherein the biotin-binding protein is non-covalently bound to a biotinylated component (e.g., tumor cell or tumor antigen). In some embodiments, the pharmaceutical composition further comprises an immunotherapy (e.g., an anti-PD-1 antibody). In specific embodiments, the tumor antigen that is biotinylated and non-covalently bound to the heat shock protein fusion is a peptide selected from Table 1 or Table 3. The pharmaceutical compositions may further comprise a pharmaceutically acceptable carrier.

The heat shock protein fusion and biotinylated components (e.g., tumor cells or tumor antigens) produced as described above may be purified to a suitable purity for use as a pharmaceutical composition. Generally, purified compositions will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a heat shock protein fusion and biotinylated components (e.g., tumor cells or tumor antigens), or a non-covalent complex of the same, using standard techniques for purification, for example, immunoaffinity chromatography, size exclusion chromatography, etc. in light of the teachings herein. Purity of a protein may be determined by a number of methods known to those of skill in the art, including for example, amino-terminal amino acid sequence analysis, gel electrophoresis and mass-spectrometry analysis.

Accordingly, provided are pharmaceutical compositions comprising the above-described heat shock protein fusion and biotinylated components (e.g., tumor cells or tumor antigens), or a non-covalent complex of the same. In one aspect, provided are pharmaceutically acceptable compositions which comprise a therapeutically-effective amount of one or more of the pharmaceutical compositions described herein, formulated together with one or more pharmaceutically acceptable carriers (additives) and/or diluents. In another aspect, in certain embodiments, the pharmaceutical compositions may be administered as such or in admixtures with pharmaceutically acceptable carriers and may also be administered in conjunction with other agents. Conjunctive (combination) therapy thus includes sequential, simultaneous and separate, or co-administration in a way that the therapeutic effects of the first administered one has not entirely disappeared when the subsequent is administered.

The heat shock protein fusion and biotinylated components (e.g., tumor cells or tumor antigens), or a non-covalent complex of the same, as described herein can be administered to a subject in a variety of ways. The routes of administration include systemic, peripheral, parenteral, enteral, topical, and transdermal (e.g., slow release polymers). Any other convenient route of administration can be used, for example, infusion or bolus injection, or absorption through epithelial or mucocutaneous linings. In addition, the compositions described herein can contain and be administered together with or without other pharmacologically acceptable components such as biologically active agents (e.g., adjuvants such as alum), surfactants (e.g., glycerides), excipients (e.g., lactose), carriers, diluents and vehicles. Furthermore, the compositions can be used ex vivo as a means of stimulating white blood cells obtained from a subject to elicit, expand and propagate antigen-specific immune cells in vitro that are subsequently reintroduced into the subject.

For pharmaceutical compositions that comprise biotinylated tumor cells, tumor cells can be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶total cells relative to an average size mouse is effective.

Administration can be accomplished using methods generally known in the art. Pharmaceutical compositions, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration.

For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine of the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

In addition, pharmaceutical compositions of the present invention can be administered to subjects or otherwise applied outside of a subject body in a biologically compatible form suitable for pharmaceutical administration. By “biologically compatible form suitable for administration in vivo” is meant a form to be administered in which any toxic effects are outweighed by the therapeutic effects. Administration of a pharmaceutical composition as described herein can be in any pharmacological form including a therapeutically active amount of an agent alone or in combination with a pharmaceutically acceptable carrier. The phrase “therapeutically-effective amount” as used herein means that amount of an agent that is effective for producing some desired therapeutic effect, e.g., cancer treatment, at a reasonable benefit/risk ratio.

Administration of a therapeutically active amount of the pharmaceutical composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

A combination dosage form or simultaneous administration of single agents can result in effective amounts of each desired modulatory agent present in the patient at the same time.

The pharmaceutical compositions described herein can be administered in a convenient manner such as by injection (subcutaneous, intravenous, etc.), oral administration, inhalation, transdermal application, or rectal administration. Depending on the route of administration, the active agent can be coated in a material to protect the agent from the action of enzymes, acids and other natural conditions which may inactivate the agent. For example, for administration of pharmaceutical compositions, by other than parenteral administration, it may be desirable to coat the pharmaceutical composition with, or co-administer the pharmaceutical composition with, a material to prevent its inactivation.

A pharmaceutical composition can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating agent such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The pharmaceutical composition may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an pharmaceutical composition of the invention in the required amount in an appropriate solvent with one or a combination of ingredients enumerated above, as required, followed by filtered sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

The compositions for administration can include a solution of the pharmaceutical composition dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of the biomarker-specific agent in these formulations can vary widely, and will be selected primarily based on fluid volumes, viscosities, body weight and the like in accordance with the particular mode of administration selected and the subject's needs.

The pharmaceutical composition may be provided in lyophilized form and rehydrated with sterile water before administration, although they are also provided in sterile solutions of known concentration. Actual methods for preparing administrable compositions will be known or apparent to those skilled in the art and are described in more detail in such publications as Remington's Pharmaceutical Science, 19th ed., Mack Publishing Company, Easton, Pa. (1995).

When the pharmaceutical composition is suitably protected, as described above, it can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active agent, use thereof in the pharmaceutical compositions is contemplated. Supplementary active agents can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active agent calculated to produce the desired therapeutic effect in association with the required pharmaceutical carrier. The specification for the dosage unit forms of the invention are dictated by, and directly dependent on, (a) the unique characteristics of the active agent and the particular therapeutic effect to be achieved, and (b) the limitations inherent in the art of compounding such an active agent for the treatment of sensitivity in individuals.

Further, a heat shock protein fusion protein can be administered by in vivo expression of a nucleic acid encoding such protein sequences into a human subject. Expression of such a nucleic acid and contact with biotinylated components (e.g., tumor cells or tumor antigens) can also be achieved ex vivo as a means of stimulating white blood cells obtained from a subject to elicit, expand and propagate antigen-specific immune cells in vitro that are subsequently reintroduced into the subject. Expression vectors suitable for directing the expression of heat shock protein fusion proteins can be selected from the large variety of vectors currently used in the field. Preferred will be vectors that are capable of producing high levels of expression as well as are effective in transducing a gene of interest. For example, recombinant adenovirus vector pJM17 (All et al., Gene Therapy 1:367-84 (1994); Berkner K. L., Biotechniques 6:616-24 1988), second generation adenovirus vectors DE1/DE4 (Wang and Finer, Nature Medicine 2:714-6 (1996)), or adeno-associated viral vector AAV/Neo (Muro-Cacho et al., J. Immunotherapy 11:231-7 (1992)) can be used. Furthermore, recombinant retroviral vectors MFG (Jaffee et al., Cancer Res. 53:2221-6 (1993)) or LN, LNSX, LNCX, LXSN (Miller and Rosman, Biotechniques 7:980-9 (1989)) can be employed. Herpes simplex virus-based vectors such as pHSV1 (Geller et al., Proc. Nat'l Acad. Sci. 87:8950-4 (1990) or vaccinia viral vectors such as MVA (Sutter and Moss. Proc. Nat'l Acad. Sci. 89:10847-51 (1992)) can serve as alternatives.

Frequently used specific expression units including promoter and 3′ sequences are those found in plasmid CDNA3 (Invitrogen), plasmid AH5, pRC/CMV (Invitrogen), pCMU II (Paabo et al., EMBO J. 5:1921-1927 (1986)), pZip-Neo SV (Cepko et al., Cell 37:1053-1062 (1984)) and pSRa (DNAX, Palo Alto, Calif.). The introduction of genes into expression units and/or vectors can be accomplished using genetic engineering techniques, as described in manuals like Molecular Cloning and Current Protocols in Molecular Biology (Sambrook, J., et al., Molecular Cloning, Cold Spring Harbor Press (1989); Ausubel, F. M. et al., Current Protocols in Molecular Biology, Greene Publishing Associates and Wiley-Interscience (1989)). A resulting expressible nucleic acid can be introduced into cells of a human subject by any method capable of placing the nucleic acid into cells in an expressible form, for example as part of a viral vector such as described above, as naked plasmid or other DNA, or encapsulated in targeted liposomes or in erythrocyte ghosts (Friedman, T., Science, 244:1275-1281 (1989); Rabinovich, N. R. et al., Science. 265:1401-1404 (1994)). Methods of transduction include direct injection into tissues and tumors, liposomal transfection (Fraley et al., Nature 370:111-117 (1980)), receptor-mediated endocytosis (Zatloukal et al., Ann. N.Y. Acad. Sci. 660:136-153 (1992)), and particle bombardment-mediated gene transfer (Eisenbraun et al., DNA & Cell. Biol. 12:791-797 (1993)).

The amount of heat shock protein fusion and biotinylated components (e.g., tumor cells or tumor antigens), or a non-covalent complex of the same, in the compositions of the present invention is an amount which produces an effective immunostimulatory response in a subject. An effective amount is an amount such that when administered, it induces an immune response. In addition, the amount of heat shock protein fusion and biotinylated components, or a non-covalent complex of the same, administered to the subject will vary depending on a variety of factors, including the heat shock protein fusion and biotinylated component employed, the size, age, body weight, general health, sex, and diet of the subject as well as on its general immunological responsiveness. Adjustment and manipulation of established dose ranges are well within the ability of those skilled in the art. For example, the amount of heat shock protein fusion, biotinylated components, or a non-covalent complex of the same, can be from about 1 microgram to about 1 gram, preferably from about 100 microgram to about 1 gram, and from about 1 milligram to about 1 gram. An effective amount of a composition comprising an expression vector is an amount such that when administered, it induces an immune response against the antigen against which the pharmaceutical composition is directed. Furthermore, the amount of expression vector administered to the subject will vary depending on a variety of factors, including the heat shock protein fusion expressed, the size, age, body weight, general health, sex, and diet of the subject, as well as on its general immunological responsiveness. Additional factors that need to be considered are the route of application and the type of vector used. For example, when prophylactic or therapeutic treatment is carried out with a viral vector containing a nucleic acid encoding heat shock protein fusion, the effective amount will be in the range of 10⁴ to 10¹² helper-free, replication-defective virus per kg body weight, preferably in the range of 10⁵ to 10¹¹ virus per kg body weight and most preferably in the range of 10⁶ to 10¹⁰ virus per kg body weight.

Determination of an effective amount of fusion protein and biotinylated components, or a non-covalent complex of the same, for inducing an immune response in a subject is well within the capabilities of those skilled in the art, especially in light of the detailed disclosure provided herein.

An effective dose can be estimated initially from in vitro assays. For example, a dose can be formulated in animal models to achieve an induction of an immune response using techniques that are well known in the art. One having ordinary skill in the art could readily optimize administration to humans based on animal data. Dosage amount and interval may be adjusted individually. For example, when used as a vaccine, the proteins and/or strains of the invention may be administered in about 1 to 3 doses for a 1-36 week period. Preferably, 3 doses are administered, at intervals of about 3-4 months, and booster vaccinations may be given periodically thereafter. Alternate protocols may be appropriate for individual patients. A suitable dose is an amount of protein or strain that, when administered as described above, is capable of raising an immune response in an immunized patient sufficient to protect the patient from the condition or infection for at least 1-2 years.

The compositions may also include adjuvants to enhance immune responses. In addition, such proteins may be further suspended in an oil emulsion to cause a slower release of the proteins in vivo upon injection. The optimal ratios of each component in the formulation may be determined by techniques well known to those skilled in the art.

Any of a variety of adjuvants may be employed in the vaccines of this invention to enhance the immune response. Most adjuvants contain a substance designed to protect the antigen from rapid catabolism, such as aluminum hydroxide or mineral oil, and a specific or nonspecific stimulator of immune responses, such as lipid A, or Bortadella pertussis. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A, quil A, SBAS1c, SBAS2 (Ling et al., 1997, Vaccine 15:1562-1567), SBAS7, Al(OH)₃ and CpG oligonucleotide (WO96/02555).

In the vaccines of the present invention, the adjuvant may induce a Th1 type immune response. Suitable adjuvant systems include, for example, a combination of monophosphoryl lipid A, preferably 3-de-O-acylated monophosphoryl lipid A (3D-MPL) together with an aluminum salt. An enhanced system involves the combination of a monophosphoryl lipid A and a saponin derivative, particularly the combination of 3D-MLP and the saponin QS21 as disclosed in WO 94/00153, or a less reactogenic composition where the QS21 is quenched with cholesterol as disclosed in WO 96/33739. Previous experiments have demonstrated a clear synergistic effect of combinations of 3D-MLP and QS21 in the induction of both humoral and Th1 type cellular immune responses. A particularly potent adjuvant formation involving QS21, 3D-MLP and tocopherol in an oil-in-water emulsion is described in WO 95/17210 and may comprise a formulation.

### Methods of Treatment

The present invention provides for both prophylactic and therapeutic methods of treating a subject at risk of (or susceptible to) a cancer or afflicted with a cancer. The cancer may be a solid or hematological cancer. The cancer may be a sarcoma or a carcinoma, e.g., a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemias, polycythemia Vera, lymphoma, multiple myeloma, Waldenstrom's macroglobulinemia, head and neck cancer, anal cancer, or heavy chain disease.

In certain embodiments, the cancer is an ovarian cancer, such as serous or epithelial papillary ovarian cancer. In some embodiments, the cancer is induced by infection of a tumor-producing virus (e.g, HPV, HCV, EBV, HIV, or Herpes virus). In certain embodiments, the cancer may be a HPV-related cancer (e.g., a Human Papilloma Virus (HPV)-induced cervical cancer, HPV-induced head and neck cancer, or HPV-induced anal cancer).

In one embodiment, the cancer is the same cancer type or has the same genetic mutations as the biotinylated tumor cells or tumor antigens. In another embodiment, the cancer is a different cancer type or has different genetic mutations from the biotinylated tumor cells or tumor antigens.

The heat shock protein fusion and biotinylated components (e.g., tumor cells or tumor antigens) described herein can be administered to a subject to induce or enhance that subject's anti-tumor immune response. The heat shock protein fusion may simply enhance the immune response (thus serving as an immunogenic composition), or confer protective immunity (thus serving as a vaccine). Accordingly, provided herein are also methods of inducing immune response using pharmaceutical compositions described herein.

a. Prophylactic Methods

In one aspect, the present invention provides a method for preventing ovarian cancer in a subject by administering to the subject an effective amount of a pharmaceutical composition comprising a heat shock protein fused to a biotin-binding protein, wherein the biotin-binding protein is non-covalently bound to a biotinylated peptide and wherein the peptide is selected from Table 1 or Table 3.

In another aspect, the present invention provides a method for preventing cancer (e.g., ovarian cancer, head and neck cancer, anal cancer, or cervical cancer) in a subject by administering to the subject an effective amount of a pharmaceutical composition comprising: (1) a heat shock protein fused to a biotin-binding protein, wherein the biotin-binding protein is non-covalently bound to a biotinylated tumor cell or a biotinylated tumor antigen (e.g., one or more peptides selected from Table 1 or Table 3); and (2) an immunotherapy (e.g., an anti-PD-1 antibody).

In still another aspect, the present invention provides a method for preventing cancer (e.g., ovarian cancer, head and neck cancer, anal cancer, or cervical cancer) in a subject, comprising conjointly administering to the subject an immunotherapy (e.g., an anti-PD-1 antibody) and an effective amount of a pharmaceutical composition comprising a heat shock protein fused to a biotin-binding protein, wherein the biotin-binding protein is non-covalently bound to a biotinylated tumor cell or a biotinylated tumor antigen (e.g., one or more peptides selected from Table 1 or Table 3).

In yet another aspect, the present invention provides a method for preventing HPV-related cancer (e.g., head and neck cancer or anal cancer) in a subject, comprising administering to the subject an effective amount of a pharmaceutical composition comprising a heat shock protein fused to a biotin-binding protein, wherein the biotin-binding protein is non-covalently bound to a biotinylated HPV virus or a biotinylated HPV viral antigen. In certain embodiments, the subject has HPV or has been exposed to HPV.

Administration of a prophylactic agent (e.g., the pharmaceutical compositions described herein) can occur prior to the manifestation of symptoms characteristic of cancer, such that a cancer is prevented or, alternatively, delayed in its progression. In certain embodiments, administration of the prophylactic agent (e.g., the pharmaceutical compositions described herein) protects the subject from recurrent cancer.

b. Therapeutic Methods

Other aspects of the present invention pertains to methods treating a subject afflicted with cancer. For example, in one aspect, the present invention provides a method for treating ovarian cancer in a subject by administering to the subject an effective amount of a pharmaceutical composition comprising a heat shock protein fused to a biotin-binding protein, wherein the biotin-binding protein is non-covalently bound to a biotinylated peptide and wherein the peptide is selected from Table 1 or Table 3.

In another aspect, the present invention provides a method for treating cancer (e.g., ovarian cancer, head and neck cancer, anal cancer, or cervical cancer) in a subject by administering to the subject an effective amount of a pharmaceutical composition comprising: (1) a heat shock protein fused to a biotin-binding protein, wherein the biotin-binding protein is non-covalently bound to a biotinylated tumor cell or a biotinylated tumor antigen (e.g., one or more peptides selected from Table 1 or Table 3); and (2) an immunotherapy (e.g., an anti-PD-1 antibody).

In still another aspect, the present invention provides a method for treating cancer (e.g., ovarian cancer, head and neck cancer, anal cancer, or cervical cancer) in a subject, comprising conjointly administering to the subject an immunotherapy (e.g., an anti-PD-1 antibody) and an effective amount of a pharmaceutical composition comprising a heat shock protein fused to a biotin-binding protein, wherein the biotin-binding protein is non-covalently bound to a biotinylated tumor cell or a biotinylated tumor antigen (e.g., one or more peptides selected from Table 1 or Table 3).

In yet another aspect, the present invention provides a method for treating HPV-related cancer (e.g., head and neck cancer or anal cancer) in a subject, comprising administering to the subject an effective amount of a pharmaceutical composition comprising a heat shock protein fused to a biotin-binding protein, wherein the biotin-binding protein is non-covalently bound to a biotinylated HPV virus or a biotinylated HPV viral antigen.

c. Combination Therapy

In some aspects, the self-assembling vaccine described herein can be administered in combination with an immunotherapy. The immunotherapy and the self-assembling vaccine may be administered concurrently or sequentially. For example, the self-assembling vaccine may be administered before, concurrently, or after the immunotherapy.

The pharmaceutical compositions described herein can also be administered in combination with untargeted therapy, e.g., chemotherapeutic agents, hormones, antiangiogens, radiolabelled, compounds, or with surgery, cryotherapy, and/or radiotherapy. The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. The foregoing examples of chemotherapeutic agents are illustrative, and are not intended to be limiting. For example, the pharmaceutical composition described herein can be administered with a therapeutically effective dose of chemotherapeutic agent. In another embodiment, the pharmaceutical composition is administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated, or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The preceding treatment methods and/or pharmaceutical compositions can be administered in conjunction with other forms of conventional therapy (e.g., standard-of-care treatments for cancer well-known to the skilled artisan), either consecutively with, pre- or post-conventional therapy. The duration and/or dose of treatment with the cancer vaccine may vary according to the particular self-assembling vaccine, or the particular combinatory therapy. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

### Kits

The present invention provides kits for expressing or administering a heat shock protein fused to a biotin-binding protein. Such kits may be comprised of nucleic acids encoding heat shock protein fused to a biotin-binding protein. The nucleic acids may be included in a plasmid or a vector, e.g., a bacterial plasmid or viral vector. Other kits comprise a heat shock protein fused to a biotin-binding protein. In some embodiments, the kits may also include an immunotherapy, such as an anti-PD-1 antibody. Furthermore, the present invention provides kits for producing and/or purifying a heat shock protein fused to a biotin-binding protein. The kits described herein may optionally include biotinylated tumor cells and/or tumor antigens. In certain embodiments, such kits may include tumor cells and/or tumor antigens, and biotinylation reagents as described herein.

The present invention provides kits for preventing and/or treating cancer in a patient. For example, a kit may comprise one or more pharmaceutical compositions as described above and optionally instructions for their use. In still other embodiments, the invention provides kits comprising one or more pharmaceutical composition and one or more devices for accomplishing administration of such compositions.

Kit components may be packaged for either manual or partially or wholly automated practice of the foregoing methods. In other embodiments involving kits, instructions for their use may be provided.

Other embodiments of the present invention are described in the following Examples. The present invention is further illustrated by the following examples which should not be construed as further limiting. The contents of all references, patents and published patent applications cited throughout this application, as well as the Figures, are incorporated herein by reference.

### EXEMPLIFICATION

### Example 1: Treatment of Ovarian Cancer

a. Study Design

Ovarian cancer patients often present late in the disease progression, with tumors that have a low number of mutations and are not easily detected by the immune system. An immunocompetent murine model of ovarian cancer was used in this study. As with human disease, the ID8 model has a low number of mutations and recapitulates the disseminated tumors, as seen in late stage patients. Previously it has been shown that anti-PD-1 treatment can provide limited benefit in the ID8 model. To maximize the potential of a SAV targeting ovarian cancer, a ID8 specific SAV was partnered with anti-PD-1 antibody therapy. Cognizant of both the advantages and challenges of treating ovarian cancer and of the ID8 model system, this approach was chosen as a “high-bar” test of the SAV cancer approach, with the objective to observe improved survival in mice treated with the vaccine alone and to potentially enhance the effect of anti-PD-1 antibody therapy.

b. Treatments and Results

In the test of the SAV cancer vaccine for ovarian cancer, peptides from both mutated proteins (neoantigens) and proteins that are overly abundant in the tumor were included (see Table 1), with the rationale to provide the immune system a broad collection of tumor targets. Mice were first injected with ovarian cancer cells and 10 days later were vaccinated with SAV containing the tumor targeting peptides. Treatment with anti-PD-1 antibody was started 3 days after vaccination and continued every 3^{rd} day until day 60 of the experiment. Mice were monitored daily, with tumor growth measured weekly for the first 4 weeks.

The study demonstrated that treatment with anti-PD-1 antibody, SAV cancer vaccine, or the combination of both, each extended the survival of tumor bearing mice as compared to control group mice treated with the either peptide alone or the MAV protein alone. Importantly, treatment with either SAV cancer vaccine alone or the vaccine in combination with anti-PD-1 antibody resulted in enhanced survival as compared to mice treated only with anti-PD-1 antibody. The most substantial improvement in survival was observed in those mice treated with the combination of SAV cancer vaccine and anti-PD-1 antibody, where 3 of 7 mice lived to beyond 100 days. In contrast, while the anti-PD-1 antibody-treated mice initially survived longer, the overall survival of the group was not improved as compared the control groups of mice. Taken together, the survival data indicate that SAV and anti-PD-1 antibody in combination can improve outcomes for ovarian cancer patients. In immunological studies of tumor infiltrating lymphocytes, it was found that administration of the SAV/anti-PD-1 antibody combination generated the highest levels of immune cell proliferation of all treatment groups and which, at least in part, contributes to the improved survival of this arm of the study compared to the mice receiving other treatments. See FIG. 2.

c. Conclusions

In this study, the presence of targetable proteins and mutations in the ID8 model of ovarian cancer was confirmed. Candidate sequences from the selected tumor targets were next identified, and peptides were designed and synthesized to construct a SAV that targets ID8 tumors. The study demonstrated the effectiveness of SAV cancer vaccine alone, and the synergistic effects of SAV cancer vaccine in combination with immunotherapy (e.g., anti-PD-1 antibody therapy). Larger groups of mice were used to provide sufficient statistical power and to generate definitive measures of the efficacy of the SAV platform in pre-clinical cancer.

### Example 2: Prophetic—Treatment of Ovarian Cancer in a Preclinical Model

The positive results from a challenging model system described in Example 1 warrant further refinement and investigation of the SAV cancer vaccine. The SAV cancer platform can be refined in several ways. First, a modified form of the MAV developed in other anti-cancer studies is used to improve stimulation of the immune system. Second, the strength and specificity of the immune response against cancer-targeting peptides delivered with the SAV platform are measured. These results are then compared to previous reports for other peptide-based approaches, and any peptides that are underperforming are identified, which guides further optimization. This evaluation of immune stimulation includes a second class of tumor-derived peptides called phospho-peptides. Though not included in the study described above, these peptides can similarly be recognized by the immune system and expand the number of targets within the tumor. The ID8 model can use a range of initial doses of cancer cells. Reduced doses of tumor cells slow the growth of the tumors. In light of the challenging nature of the ID8 model, mice injected with a reduced quantity of cancerous cells are evaluated. In previous experiments, it has been shown that reduce numbers of cells lead to a slower growing tumor and longer overall survival. The advantage of this approach is that the vaccine has more time to fight against the tumor and a lower overall burden to combat. Finally, a large-scale study of the refined SAV cancer vaccine is conducted, which is sufficiently powered to provide robust statistics on survival and assays are included to generate definitive data on the performance of the SAV platform.

Following the incorporation of refinements, a large-scale study that includes a robust description of the changes in the immune function and tumor biology in response to treatment as well as a sufficiently powered survival arms to provide adequate statistics is performed. A detailed description of the immune system using CyTOF is included in the full study, which offers a broad and detailed assessment that is not available using traditional flow cytometry. Tumors that can be later interrogated using RNAseq are banked, which provides information on the behavior of both cancer cells and immune cells within the tumor. Banked tumors can also be utilized to conduct layered imaging analysis to map the distribution of drugs and immune cells in the tumor.

### Example 3: Prophetic—Treatment of HPV-Related Cancers

Murine model systems that are more sensitive to immune therapy and vaccination are also investigated. Three model systems are evaluated. The first is a model of Human Papilloma Virus (HPV) induced cervical cancer, which provides a number of well-established HPV viral antigens and is a second gynecological cancer. Effects of the SAVs for preventing and/or treating HPV-related head and neck cancer and anal cancer are also evaluated.

TC-1 model is made using HPV16. Vaccines are designed to target E6 and/or E7 epitopes. The peptides for C57 and Balb/c mice are different, as C57 and Balb/c mice have different MHC alleles (see Table 4). C57BL/6 female mice are used. Epitopes from HPV E6 and E7 proteins are selected from consensus sequences in the literature (see Table 5). No optimization by 21^{st} Century for production is required.

TABLE 4

| Mouse Strains | MHC Haplotype | H-2K | H-2D | H-2L |
|---|---|---|---|---|
| BALB/CJ | d | d | d | d |
| C57BL/6 | b | b | b | null |

The peptides used in the study include:

E6
(SEQ ID NO: 3)
QLLRREVYDFAFRDLC
E7
(SEQ ID NO: 4)
GQAEPDRAHYNIVTFCCKCD

TABLE 5
A list of additional peptides used in the study

| Mouse | | | | SEQ ID |
|---|---|---|---|---|
| Strain | Publication | Epitope | Sequence | NO |
| C57BL/6 | PMID | HPV16 | QLLRREVYDF | 5 |
| | 17291642 | E6⁴³⁻⁵⁷ | AFRDL | |
| C57BL/6 | PMID | HPV16 | QLLRREVYDF | 5 |
| | 26351680 | E6⁴³⁻⁵⁷ | AFRDL | |
| C57BL/6 | PMID | HPV16 | VYDFAFRDLC | 6 |
| | 26351680 | E6⁴⁹⁻⁵⁸ | | |
| C57BL/6 | PMID | HPV16 | VYDFAFRDLC | 6 |
| | 17291642 | E6⁴⁹⁻⁵⁸ | | |
| C57BL/6 | PMID | HPV16 | QLLRREVYDF | 5 |
| | 30054333 | E6⁴³⁻⁵⁷ | AFRDL | |
| C57BL/6 | PMID | HPV16 | QAEPDRAHVY | 7 |
| | 26351680 | E7⁴⁴⁻⁶² | NIVTFCCKCD | |
| C57BL/ | PMID | HPV16 | GQAEPDRAHY | 8 |
| 6jico | 25888578 | E7⁴³⁻⁷⁷ | NIVTFCCKCD | |
| | | | STLRLCVQST | |
| | | | HVDIR | |
| C57BL/6 | PMID | HPV16 | QAEPDRAHVY | 7 |
| | 17291642 | E7⁴⁴⁻⁶² | NIVTFCCKCD | |
| C57BL/6 | PMID | HPV16 | RAHVYNIVTF | 9 |
| | 17291642 | E7⁴⁹⁻⁵⁷ | | |
| C57BL/6 | PMID | HPV16 | RAHVYNIVTF | 9 |
| | 30054333 | E7⁴⁹⁻⁵⁷ | | |
| C57BL/6 | PMID | HPV16 | GQAEPDRAHV | 10 |
| | 26949512 | E7⁴³⁻⁶² | YNIVTFCCKC | |
| | | | D | |

This list comes from the literature and may not represent an exhaustive search. They are consistent with the predictions generated using publicly available MHC epitope selection tools. They reflect consensus sequences used by numerous studies for HPV driven tumor models.

The sequences used overlap with sequences applicable to human tumors driven by HPV-16. Those sequences share approximately 70% homology with HPV 31, 33, 45, 58, and 73. A distinct set of peptides can be used for human studies. For example, the human HLA-DR11 allele binds the peptide spanning HPV16 E6 amino acid (AA) 52-62, while we used HPV16 E6 AA43-57.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A pharmaceutical composition comprising a heat shock protein fused to a biotin-binding protein, wherein the biotin-binding protein is non-covalently bound to a biotinylated peptide, and wherein the peptide:
(1) binds to a MHC class I molecule; and
(2) has less than 100% homology to an autologous native sequence and/or a native microbiome sequence.

2. The pharmaceutical composition of claim 1, wherein the biotin-binding protein is selected from the group consisting of avidin, streptavidin, and neutravidin.

3. The pharmaceutical composition of claim 1 or 2, wherein the biotin-binding protein has an amino acid sequence that is at least 80%, 85%, 90%, 95%, or 99% identical to avidin or streptavidin.

4. The pharmaceutical composition of any one claims 1-3, wherein the heat shock protein is a mammalian heat shock protein or a bacterial heat shock protein.

5. The pharmaceutical composition of any one of claims 1-4, wherein the heat shock protein is a member of the hsp70 family.

6. The pharmaceutical composition of any one of claims 1-5, wherein the heat shock protein is or is derived from MTB-HSP70; optionally wherein the heat shock protein has an amino acid sequence that is at least 80%, 85%, 90%, 95%, or 99% identical to SEQ ID NO: 1 or SEQ ID NO: 2.

7. The pharmaceutical composition of any one of claims 1-6, wherein the peptide has a length of 5-50 amino acids; optionally the peptide has a length of 8-12 amino acids.

8. The pharmaceutical composition of any one of claims 1-7, wherein the peptide is one or more peptides selected from Table 1 (on page 33).

9. The pharmaceutical composition of any one of claims 1-8, further comprising a pharmaceutically acceptable carrier.

10. The pharmaceutical composition of any one of claims 1-9, wherein the pharmaceutical composition increases survival rate of subjects afflicted with ovarian cancer.

11. The pharmaceutical composition of claim 10, wherein the ovarian cancer is serous or epithelial papillary ovarian cancer.

12. The pharmaceutical composition of any one of claims 1-11, wherein the pharmaceutical composition increases an immune response.

13. The pharmaceutical composition of any one of claims 1-12, wherein the pharmaceutical composition increases proliferation of immune cells.

14. A method for producing a pharmaceutical composition of any one of claims 1-13, comprising contacting a heat shock protein fused to a biotin-binding protein with a biotinylated peptide, sufficient to form a non-covalent complex of the heat shock protein and the biotinylated peptide, wherein the peptide:
(1) binds to a MHC class I molecule; and
(2) has less than 100% homology to an autologous native sequence and/or a native microbiome sequence.

15. A method of inducing an immune response in a subject, comprising administering to the subject an effective amount of a pharmaceutical composition of any one of claims 1-13.

16. A method of preventing and/or treating ovarian cancer in a subject, comprising administering to the subject an effective amount of a pharmaceutical composition comprising a heat shock protein fused to a biotin-binding protein, wherein the biotin-binding protein is non-covalently bound to a biotinylated peptide and wherein the peptide:
(1) binds to a MHC class I molecule; and
(2) has less than 100% homology to an autologous native sequence and/or a native microbiome sequence.

17. The method of claim 16, wherein the biotin-binding protein is selected from the group consisting of avidin, streptavidin, and neutravidin.

18. The method of claim 16 or 17, wherein the biotin-binding protein has an amino acid sequence that is at least 80%, 85%, 90%, 95%, or 99% identical to avidin or streptavidin.

19. The method of any one of claims 16-18, wherein the heat shock protein is a mammalian heat shock protein or a bacterial heat shock protein.

20. The method of any one of claims 16-19, wherein the heat shock protein is a member of the hsp70 family.

21. The method of any one of claims 16-20, wherein the heat shock protein is or is derived from MTB-HSP70; optionally wherein the heat shock protein has an amino acid sequence that is at least 80%, 85%, 90%, 95%, or 99% identical to SEQ ID NO: 1 or SEQ ID NO: 2.

22. The method of any one of claims 16-21, wherein the peptide has a length of 5-50 amino acids; optionally the peptide has a length of 8-12 amino acids.

23. The method of any one of claims 16-22, wherein the peptide is one or more peptides selected from Table 1 (on page 33).

24. The method of any one of claims 16-23, wherein the pharmaceutical composition further comprises a pharmaceutically acceptable carrier.

25. The method of any one of claims 16-24, wherein the pharmaceutical composition increases survival rate of subjects afflicted with ovarian cancer.

26. The method of any one of claims 16-25, wherein the pharmaceutical composition increases an immune response in the subject.

27. The method of any one of claims 16-26, wherein the pharmaceutical composition increases proliferation of immune cells.

28. The method of any one of claims 16-27, wherein the method is a method of treating ovarian cancer.

29. The method of any one of claims 16-28, wherein the ovarian cancer is serous or epithelial papillary ovarian cancer.

30. The method of any one of claims 16-29, wherein the pharmaceutical composition is administered to the subject as a non-covalent complex.

31. A pharmaceutical composition comprising:
(1) a heat shock protein fused to a biotin-binding protein, wherein the biotin-binding protein is non-covalently bound to a biotinylated tumor cell or a biotinylated tumor antigen; and
(2) an immunotherapy.

32. The pharmaceutical composition of claim 31, wherein the biotin-binding protein is selected from the group consisting of avidin, streptavidin, and neutravidin.

33. The pharmaceutical composition of claim 30 or 31, wherein the biotin-binding protein has an amino acid sequence that is at least 80%, 85%, 90%, 95%, or 99% identical to avidin or streptavidin.

34. The pharmaceutical composition of any one of claims 31-33, wherein the heat shock protein is a mammalian heat shock protein or a bacterial heat shock protein.

35. The pharmaceutical composition of any one of claims 31-34, wherein the heat shock protein is a member of the hsp70 family.

36. The pharmaceutical composition of any one of claims 31-35, wherein the heat shock protein is or is derived from MTB-HSP70.

37. The pharmaceutical composition of any one of claims 31-36, wherein the heat shock protein has an amino acid sequence that is at least 80%, 85%, 90%, 95%, or 99% identical to SEQ ID NO: 1 or SEQ ID NO: 2.

38. The pharmaceutical composition of any one of claims 31-37, wherein the biotin-binding protein is non-covalently bound to a biotinylated tumor cell; and the biotinylated tumor cell expresses an antigen on its surface.

39. The pharmaceutical composition of any one of claims 31-38, wherein the tumor cell is non-replicative.

40. The pharmaceutical composition of any one of claims 31-39, wherein the tumor cell is non-replicative due to irradiation.

41. The pharmaceutical composition of any one of claims 31-40, wherein the biotinylated tumor cell is a biotinylated sarcoma cell or a biotinylated carcinoma cell.

42. The pharmaceutical composition of any one of claims 31-41, wherein the biotinylated tumor cell is a biotinylated fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemia, polycythemia Vera, lymphoma, multiple myeloma, Waldenstrom's macroglobulinemia, head and neck cancer, anal cancer, or heavy chain disease cell.

43. The pharmaceutical composition of any one of claims 31-42, wherein the biotinylated tumor cell is a biotinylated ovarian cancer cell; optionally wherein the biotinylated ovarian cancer cell is a biotinylated serous or epithelial papillary ovarian cancer cell.

44. The pharmaceutical composition of any one of claims 31-42, wherein the biotinylated tumor cell is a biotinylated HPV-related cancer cell; optionally wherein the biotinylated HPV-related cancer cell is a biotinylated HPV-induced head and neck cancer cell, a biotinylated HPV-induced cervical cancer cell, or a biotinylated HPV-induced anal cancer cell.

45. The pharmaceutical composition of any one of claims 31-37, wherein the biotin-binding protein is non-covalently bound to a biotinylated tumor antigen.

46. The pharmaceutical composition of any one of claims 31-37 and claim 45, wherein the tumor antigen is a protein that is overexpressed by a tumor cell, or an immunogenic fragment thereof.

47. The pharmaceutical composition of any one of claims 31-37 and claims 45-46, wherein the tumor antigen is a protein that is specifically mutated in a tumor cell, or an immunogenic fragment thereof.

48. The pharmaceutical composition of any one of claims 31-37 and claims 45-47, wherein the tumor antigen comprises a whole or partial inactivated tumor-producing virus; or comprises a protein or an immunogenic fragment thereof that is derived from a tumor-producing virus; optionally wherein the tumor-producing virus is a Human Papillomavirus (HPV), Hepatitis C Virus (HCV), Epstein-Barr Virus (EBV), Human Immunodeficiency Virus (HIV), or Herpes virus.

49. The pharmaceutical composition of any one of claims 31-37 and claims 45-48, wherein the tumor antigen is tumor-derived phospho-peptides.

50. The pharmaceutical composition of any one of claims 31-37 and claims 45-49, wherein the tumor antigen is capable of eliciting an immune response.

51. The pharmaceutical composition of any one of claims 31-37 and claim 45-50, wherein the tumor antigen is derived from a sarcoma cell or a carcinoma cell.

52. The pharmaceutical composition of any one of claims 31-37 and claims 45-51, wherein the tumor antigen is derived from a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemias, polycythemia Vera, lymphoma, multiple myeloma, Waldenstrom's macroglobulinemia, head and neck cancer, anal cancer, or heavy chain disease cell.

53. The pharmaceutical composition of any one of claims 31-37 and claims 45-52, wherein the tumor antigen is derived from an ovarian cancer cell.

54. The pharmaceutical composition of any one of claims 31-37 and claims 45-53, wherein the tumor antigen is derived from a serous or epithelial papillary ovarian cancer cell.

55. The pharmaceutical composition of any one of claims 31-37 and claims 45-54, wherein the tumor antigen is one or more peptides selected from Table 1.

56. The pharmaceutical composition of any one of claims 31-37 and claims 45-52, wherein the tumor antigen is derived from a HPV-related cancer cell; optionally wherein the HPV-related cancer cell is a HPV-induced head and neck cancer cell, a HPV-induced cervical cancer cell, or a HPV-induced anal cancer cell.

57. The pharmaceutical composition of any one of claims 31-56, wherein the immunotherapy inhibits an immune checkpoint.

58. The pharmaceutical composition of any one of claims 31-57, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; optionally wherein the immune checkpoint is PD1 or PD-L1.

59. The pharmaceutical composition of any one of claims 31-58, wherein the immunotherapy is an anti-PD-1 antibody.

60. The pharmaceutical composition of any one of claims 31-56, wherein the immunotherapy is an immune modulatory agent selected from the group consisting of a CXCR4/CXCR7 antagonist, a Jak/stat inhibitor, and a near infrared laser immunomodulation of skin associated immune cell.

61. The pharmaceutical composition of any one of claims 31-60, further comprising a pharmaceutically acceptable carrier.

62. The pharmaceutical composition of any one of claims 31-61, wherein the pharmaceutical composition increases survival rate of subjects afflicted with cancer.

63. The pharmaceutical composition of any one of claims 31-61, wherein the pharmaceutical composition increases an immune response.

64. The pharmaceutical composition of any one of claims 31-62, wherein the pharmaceutical composition increases proliferation of immune cells.

65. A method of inducing an immune response in a subject, comprising administering to the subject an effective amount of a pharmaceutical composition of any one of claims 31-64.

66. A method of preventing and/or treating cancer in a subject, comprising administering to the subject an effective amount of a pharmaceutical composition of any one of claims 31-64.

67. The method of claim 66, wherein the biotinylated tumor cell or the biotinylated tumor antigen in the pharmaceutical composition of any one of claims 31-64 is derived from the same type of cancer as the cancer to be prevented and/or treated.

68. The method of claim 66 or 67, wherein the method is a method of treating cancer.

69. The method of any one of claims 66-68, wherein the cancer is ovarian cancer; optionally wherein the ovarian cancer is serous or epithelial papillary ovarian cancer.

70. The method of any one of claims 66-69, wherein the cancer is induced by infection of a tumor-producing virus; optionally wherein the tumor-producing virus is a Human Papillomavirus (HPV), Hepatitis C Virus (HCV), Epstein-Barr Virus (EBV), Human Immunodeficiency Virus (HIV), or Herpes virus.

71. The method of any one of claims 66-70, wherein the cancer a HPV-related cancer.

72. The method of claim 71, wherein the HPV-related cancer is a HPV-induced cervical cancer, HPV-induced head and neck cancer, or HPV-induced anal cancer.

73. The method of any one of claims 66-72, wherein the method further comprises a cancer therapy selected from the group consisting of radiation, a radiosensitizer, a chemotherapy, and a second immunotherapy; optionally wherein the second immunotherapy is an immune checkpoint inhibitor or an immune modulator selected from a CXCR4/CXCR7 antagonist, a Jak/stat inhibitor, or a near infrared laser immunomodulation of skin associated immune cell.

74. A method of preventing and/or treating cancer in a subject, comprising conjointly administering to the subject an immunotherapy and an effective amount of a pharmaceutical composition comprising a heat shock protein fused to a biotin-binding protein, wherein the biotin-binding protein is non-covalently bound to a biotinylated tumor cell or a biotinylated tumor antigen.

75. The method of claim 74, wherein the immunotherapy and the pharmaceutical composition are administered concurrently or sequentially.

76. The method of claim 74 or 75, wherein the pharmaceutical composition is administered before the immunotherapy.

77. The method of any one of claims 74-76, wherein the biotin-binding protein is selected from the group consisting of avidin, streptavidin, and neutravidin.

78. The method of any one of claims 74-77, wherein the biotin-binding protein has an amino acid sequence that is at least 80%, 85%, 90%, 95%, or 99% identical to avidin or streptavidin.

79. The method of any one of claims 74-78, wherein the heat shock protein is a mammalian heat shock protein or a bacterial heat shock protein.

80. The method of any one of claims 74-79, wherein the heat shock protein is a member of the hsp70 family.

81. The method of any one of claims 74-80, wherein the heat shock protein is or is derived from MTB-HSP70.

82. The method of any one of claims 74-81, wherein the heat shock protein has an amino acid sequence that is at least 80%, 85%, 90%, 95%, or 99% identical to SEQ ID NO: 1 or SEQ ID NO: 2.

83. The method of any one of claims 74-82, wherein the biotin-binding protein is non-covalently bound to a biotinylated tumor cell; and the biotinylated tumor cell expresses an antigen on its surface.

84. The method of any one of claims 74-83, wherein the tumor cell is non-replicative.

85. The method of any one of claims 74-84, wherein the tumor cell is non-replicative due to irradiation.

86. The method of any one of claims 74-85, wherein the biotinylated tumor cell is a biotinylated sarcoma cell or a biotinylated carcinoma cell.

87. The method of any one of claims 74-86, wherein the biotinylated tumor cell is a biotinylated fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemia, polycythemia Vera, lymphoma, multiple myeloma, Waldenstrom's macroglobulinemia, head and neck cancer, anal cancer, or heavy chain disease cell.

88. The method of any one of claims 74-87, wherein the biotinylated tumor cell is a biotinylated ovarian cancer cell; optionally wherein the biotinylated ovarian cancer cell is a biotinylated serous or epithelial papillary ovarian cancer.

89. The method of any one of claims 74-87, wherein the biotinylated tumor cell is a biotinylated HPV-related cancer cell; optional wherein the HPV-related cancer cell is a HPV-induced head and neck cancer cell, HPV-induced cervical cancer cell, or HPV-induced anal cancer cell.

90. The method of any one of claims 74-82, wherein the biotin-binding protein is non-covalently bound to a biotinylated tumor antigen.

91. The method of any one of claims 74-82 and 90, wherein the tumor antigen is a protein that is overexpressed by a tumor cell, or an immunogenic fragment thereof.

92. The method of any one of claims 74-82 and 90-91, wherein the tumor antigen is a protein that is specifically mutated in a tumor cell, or an immunogenic fragment thereof.

93. The method of any one of claims 74-82 and 90-92, wherein the tumor antigen comprises a whole or partial inactivated tumor-producing virus; or comprises a protein or an immunogenic fragment thereof that is derived from a tumor-producing virus; optionally wherein the tumor-producing virus is a HPV, HCV, EBV, HIV, or Herpes virus.

94. The method of any one of claims 74-82 and 90-93, wherein the tumor antigen is tumor-derived phospho-peptides.

95. The method of any one of claims 74-82 and 90-94, wherein the tumor antigen is capable of eliciting an immune response.

96. The method of any one of claims 74-82 and 90-95, wherein the tumor antigen is derived from a sarcoma cell or a carcinoma cell.

97. The method of any one of claims 74-82 and 90-96, wherein the tumor antigen is derived from a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewings tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, Sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, Small cell lung carcinoma, bladder carcinoma, epithelial carcinoma, glioma, astrocytoma, medulloblastoma, cranio pharyngioma, ependymoma, pinealoma, hemangioblastoma, acoustic neuroma, oligodendroglioma, meningioma, melanoma, neuroblastoma, retinoblastoma, leukemias, polycythemia Vera, lymphoma, multiple myeloma, Waldenstrom's macroglobulinemia, head and neck cancer, anal cancer, or heavy chain disease cell.

98. The method of any one of claims 74-82 and 90-97, wherein the tumor antigen is derived from an ovarian cancer cell.

99. The method of any one of claims 74-82 and 90-98, wherein the tumor antigen is derived from a serous or epithelial papillary ovarian cancer cell.

100. The method of any one of claims 74-82 and 90-99, wherein the tumor antigen is one or more peptides selected from Table 1.

101. The method of any one of claims 74-82 and 90-97, wherein the tumor antigen is derived from a HPV-related cancer cell; optionally wherein the HPV-related cancer cell is a HPV-induced head and neck cancer cell, a HPV-induced cervical cancer cell, or a HPV-induced anal cancer cell.

102. The method of any one of claims 74-101, wherein the immunotherapy inhibits an immune checkpoint.

103. The method of any one of claims 74-102, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; optionally wherein the immune checkpoint is PD1 or PD-L1.

104. The method of any one of claims 74-103, wherein the immunotherapy is an anti-PD-1 antibody.

105. The method of any one of claims 74-101, wherein the immunotherapy is an immune modulatory agent selected from the group consisting of a CXCR4/CXCR7 antagonist, a Jak/stat inhibitor, and a near infrared laser immunomodulation of skin associated immune cell.

106. The method of any one of claims 74-105, wherein the pharmaceutical composition further comprises a pharmaceutically acceptable carrier.

107. The method of any one of claims 74-106, wherein the method increases survival rate of subjects afflicted with cancer.

108. The method of any one of claims 74-107, wherein the method increases an immune response.

109. The method of any one of claims 74-108, wherein the method increases immune cell proliferation.

110. The method of any one of claims 74-109, wherein the biotinylated tumor cell or the biotinylated tumor antigen in the pharmaceutical composition is derived from the same type of cancer as the cancer to be prevented or treated.

111. The method of any one of claims 74-110, wherein the method is a method of treating cancer.

112. The method of any one of claims 74-111, wherein the cancer is ovarian cancer; optionally wherein the ovarian cancer is serous or epithelial papillary ovarian cancer.

113. The method of any one of claims 74-111, wherein the cancer is induced by infection of a tumor-producing virus; optionally the tumor-producing virus is a HPV, HCV, EBV, HIV, or Herpes virus.

114. The method of any one of claims 74-111, wherein the cancer a HPV-related cancer.

115. The method of claim 114, wherein the HPV-related cancer is a HPV-induced cervical cancer, a HPV-induced head and neck cancer, and a HPV-induced anal cancer.

116. The method of any one of claims 74-115, wherein the method further comprises a cancer therapy selected from the group consisting of radiation, a radiosensitizer, and a chemotherapy.

117. A method of preventing and/or treating HPV-related cancer in a subject, comprising administering to the subject an effective amount of a pharmaceutical composition comprising a heat shock protein fused to a biotin-binding protein, wherein the biotin-binding protein is non-covalently bound to a biotinylated HPV virus or a biotinylated HPV viral antigen.

118. The method of claim 117, wherein the biotin-binding protein is selected from the group consisting of avidin, streptavidin, and neutravidin.

119. The method of claim 117 or 118, wherein the biotin-binding protein has an amino acid sequence that is at least 80%, 85%, 90%, 95%, or 99% identical to avidin or streptavidin.

120. The method of any one of claims 117-119, wherein the heat shock protein is a mammalian heat shock protein or a bacterial heat shock protein.

121. The method of any one of claims 117-120, wherein the heat shock protein is a member of the hsp70 family.

122. The method of any one of claims 117-121, wherein the heat shock protein is or is derived from MTB-HSP70.

123. The method of any one of claims 117-122, wherein the heat shock protein has an amino acid sequence that is at least 80%, 85%, 90%, 95%, or 99% identical to SEQ ID NO: 1 or SEQ ID NO: 2.

124. The method of any one of claims 117-123, wherein the pharmaceutical composition is a vaccine.

125. The method of any one of claims 117-124, wherein the pharmaceutical composition further comprises a pharmaceutically acceptable carrier.

126. The method of any one of claims 117-125, wherein the pharmaceutical composition increases survival rate of subjects afflicted with HPV-related cancer.

127. The method of any one of claims 117-126, wherein the pharmaceutical composition increases an immune response in the subject.

128. The method of any one of claims 117-127, wherein the pharmaceutical composition increases proliferation of immune cells.

129. The method of any one of claims 117-128, wherein the method is a method of treating HPV-related cancer.

130. The method of any one of claims 117-129, wherein the HPV-related cancer is head and neck cancer or anal cancer.

131. The method of any one of claims 117-130, wherein the biotin-binding protein is non-covalently bound to a biotinylated HPV virus; and the biotinylated HPV virus expresses an antigen.

132. The method of any one of claims 117-131, wherein the HPV virus is a whole or partial inactivated HPV virus.

133. The method of any one of claims 117-130, wherein the biotin-binding protein is non-covalently bound to a biotinylated HPV viral antigen.

134. The method of claim 133, wherein the biotinylated HPV viral antigen is biotinylated E6 protein, biotinylated E7 protein, or a biotinylated immunogenic fragment thereof.

135. The method of claim 133 or 134, wherein the biotinylated HPV viral antigen is selected from Table 3.

136. The method of any one of claims 117-135, wherein the pharmaceutical composition is administered to the subject as a non-covalent complex.

137. A pharmaceutical composition comprising:
(1) a heat shock protein fused to a biotin-binding protein, wherein the biotin-binding protein is non-covalently bound to a biotinylated HPV virus or a biotinylated HPV viral antigen; and
(2) an immunotherapy.

138. The pharmaceutical composition of claim 137, wherein the biotin-binding protein is selected from the group consisting of avidin, streptavidin, and neutravidin.

139. The pharmaceutical composition of claim 137 or 138, wherein the biotin-binding protein has an amino acid sequence that is at least 80%, 85%, 90%, 95%, or 99% identical to avidin or streptavidin.

140. The pharmaceutical composition of any one of claims 137-139, wherein the heat shock protein is a mammalian heat shock protein or a bacterial heat shock protein.

141. The pharmaceutical composition of any one of claims 137-140, wherein the heat shock protein is a member of the hsp70 family.

142. The pharmaceutical composition of any one of claims 137-141, wherein the heat shock protein is or is derived from MTB-HSP70.

143. The pharmaceutical composition of any one of claims 137-142, wherein the heat shock protein has an amino acid sequence that is at least 80%, 85%, 90%, 95%, or 99% identical to SEQ ID NO: 1 or SEQ ID NO: 2.

144. The pharmaceutical composition of any one of claims 137-143, wherein the biotin-binding protein is non-covalently bound to a biotinylated HPV virus; and the biotinylated HPV virus expresses an antigen.

145. The pharmaceutical composition of any one of claims 137-144, wherein the HPV virus is a whole or partial inactivated HPV virus.

146. The pharmaceutical composition of any one of claims 137-143, wherein the biotin-binding protein is non-covalently bound to a biotinylated HPV viral antigen.

147. The pharmaceutical composition of claim 146, wherein the biotinylated HPV viral antigen is biotinylated E6 protein, biotinylated E7 protein, or a biotinylated immunogenic fragment thereof.

148. The pharmaceutical composition of claim 146 or 147, wherein the biotinylated HPV viral antigen is selected from Table 3.

149. The pharmaceutical composition of any one of claims 137-148, wherein the immunotherapy inhibits an immune checkpoint.

150. The pharmaceutical composition of any one of claims 137-149, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; optionally wherein the immune checkpoint is PD1 or PD-L1.

151. The pharmaceutical composition of any one of claims 137-150, wherein the immunotherapy is an anti-PD-1 antibody.

152. The pharmaceutical composition of any one of claims 137-148, wherein the immunotherapy is an immune modulatory agent selected from the group consisting of a CXCR4/CXCR7 antagonist, a Jak/stat inhibitor, and a near infrared laser immunomodulation of skin associated immune cell.

153. The pharmaceutical composition of any one of claims 137-152, further comprising a pharmaceutically acceptable carrier.

154. The pharmaceutical composition of any one of claims 137-153, wherein the pharmaceutical composition increases survival rate of subjects afflicted with HPV-related cancer.

155. The pharmaceutical composition of claim 154, wherein the HPV-related cancer is head and neck cancer or anal cancer.

156. The pharmaceutical composition of any one of claims 137-155, wherein the pharmaceutical composition increases an immune response.

157. The pharmaceutical composition of any one of claims 137-156, wherein the pharmaceutical composition increases proliferation of immune cells.

158. A method of inducing an immune response in a subject, comprising administering to the subject an effective amount of a pharmaceutical composition of any one of claims 137-157.

159. A method of preventing and/or treating HPV-related cancer in a subject, comprising administering to the subject an effective amount of a pharmaceutical composition of any one of claims 137-157.

160. The method of claim 159, wherein the method is a method of treating HPV-related cancer.

161. The method of any one of claim 159 or 160, wherein the HPV-related cancer is head and neck cancer or anal cancer.

162. The method of any one of claims 159-161, wherein the method further comprises a cancer therapy selected from the group consisting of radiation, a radiosensitizer, a chemotherapy, and a second immunotherapy; optionally wherein the second immunotherapy is an immune checkpoint inhibitor or an immune modulator selected from a CXCR4/CXCR7 antagonist, a Jak/stat inhibitor, or a near infrared laser immunomodulation of skin associated immune cell.

163. A method of preventing and/or treating HPV-related cancer in a subject, comprising conjointly administering to the subject an immunotherapy and an effective amount of a pharmaceutical composition comprising a heat shock protein fused to a biotin-binding protein, wherein the biotin-binding protein is non-covalently bound to a biotinylated HPV virus or a biotinylated HPV viral antigen.

164. The method of claim 163, wherein the immunotherapy and the pharmaceutical composition are administered concurrently or sequentially.

165. The method of claim 163 or 164, wherein the pharmaceutical composition is administered before the immunotherapy.

166. The method of any one of claims 163-165, wherein the biotin-binding protein is selected from the group consisting of avidin, streptavidin, and neutravidin.

167. The method of any one of claims 163-166, wherein the biotin-binding protein has an amino acid sequence that is at least 80%, 85%, 90%, 95%, or 99% identical to avidin or streptavidin.

168. The method of any one of claims 163-167, wherein the heat shock protein is a mammalian heat shock protein or a bacterial heat shock protein.

169. The method of any one of claims 163-168, wherein the heat shock protein is a member of the hsp70 family.

170. The method of any one of claims 163-169, wherein the heat shock protein is or is derived from MTB-HSP70.

171. The method of any one of claims 163-170, wherein the heat shock protein has an amino acid sequence that is at least 80%, 85%, 90%, 95%, or 99% identical to SEQ ID NO: 1 or SEQ ID NO: 2.

172. The method of any one of claims 163-171, wherein the biotin-binding protein is non-covalently bound to a biotinylated HPV virus; and the biotinylated HPV virus expresses an antigen.

173. The method of any one of claims 163-172, wherein the HPV virus is a whole or partial inactivated HPV virus.

174. The method of any one of claims 163-171, wherein the biotin-binding protein is non-covalently bound to a biotinylated HPV viral antigen.

175. The method of claim 174, wherein the biotinylated HPV viral antigen is biotinylated E6 protein, biotinylated E7 protein, or a biotinylated immunogenic fragment thereof.

176. The method of claim 174 or 175, wherein the biotinylated HPV viral antigen is selected from Table 3.

177. The method of any one of claims 163-176, wherein the immunotherapy inhibits an immune checkpoint.

178. The method of any one of claims 163-177, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; optionally wherein the immune checkpoint is PD1 or PD-L1.

179. The method of any one of claims 163-178, wherein the immunotherapy is an anti-PD-1 antibody.

180. The method of any one of claims 163-176, wherein the immunotherapy is an immune modulatory agent selected from the group consisting of a CXCR4/CXCR7 antagonist, a Jak/stat inhibitor, and a near infrared laser immunomodulation of skin associated immune cell.

181. The method of any one of claims 163-180, further comprising a pharmaceutically acceptable carrier.

182. The method of any one of claims 163-181, wherein the pharmaceutical composition increases survival rate of subjects.

183. The method of any one of claims 163-182, wherein the pharmaceutical composition increases an immune response.

184. The method of any one of claims 163-183, wherein the pharmaceutical composition increases proliferation of immune cells.

185. The method of any one of claims 163-184, wherein the method is a method of treating HPV-related cancer.

186. The method of any one of claims 163-185 wherein the HPV-related cancer is head and neck cancer or anal cancer.

187. The method of any one of claims 163-186, wherein the method further comprises a cancer therapy selected from the group consisting of radiation, a radiosensitizer, and a chemotherapy.
